(19) United States
(12) Patent Application Publication
(10) Pub. No.: US 2025/0303201 A1
(43) Pub. Date: Oct. 2, 2025

(54) Ultrasound Probe With Offset Balloon(s)

(71) Applicant: Profound Medical Inc., Mississauga (CA)

(72) Inventors: Christopher Driver, Oakville (CA); Samantha Lee, Mississauga (CA); Hooman Reza Zadeh Tabatabai, Richmond Hill (CA); Yifan Yang, Maple (CA)

(21) Appl. No.: 19/098,322

(22) Filed: Apr. 2, 2025

Related U.S. Application Data
(60) Provisional application No. 63/573,055, filed on Apr. 2, 2024.
(60) Provisional application No. 63/633,117, filed on Apr. 12, 2024.

(51) Int. Cl. A61N 7/02; A61M 25/10

(52) U.S. Cl. CPC A61N 7/022; A61M 25/1011; A61M 2025/1047; A61M 2025/105; A61M 2205/0205; A61M 2205/0222; A61M 2205/0238; A61M 2210/1085

(57) ABSTRACT

An ultrasound probe includes a shaft having a proximal end, a tip, and a length measured between the proximal end and the tip; a plurality of channels defined in the shaft including an ultrasound channel and a balloon channel; ultrasound transducer(s) disposed in the ultrasound channel; and an offset balloon attached to the shaft at a predetermined distance from the ultrasound transducer(s), the offset balloon fluidly coupled to the balloon channel. In an inflated state the offset balloon has an inflated size that is larger than a target anatomical opening such that an inflated offset balloon is configured to mechanically engage the target anatomical opening when the shaft is inserted or retracted with respect to the target anatomical opening. The ultrasound transducer(s) is/are aligned with respect to a target volume when the inflated offset balloon mechanically engages the target anatomical opening.

### CROSS-REFERENCE TO RELATED APPLICATIONS

This application claims priority to U.S. Provisional Application No. 63/633,117, titled “Ultrasound Probe With Distal Balloon,” filed Apr. 12, 2024 and to U.S. Provisional Application No. 63/573,055, titled “Fluid Collection Ring for Thermal Therapy Device,” filed on Apr. 2, 2024, which are hereby incorporated by reference.

### TECHNICAL FIELD

This application relates generally to ultrasound therapy devices.

### BACKGROUND

Ultrasound therapy devices are used to treat a variety of conditions including benign prostatic hyperplasia (BHP), calcifications such as bladder stones and kidney stones, and tumors. For some conditions, an ultrasound probe is inserted into the body to place the ultrasound transducers close to the target region. Imaging is typically used to align the ultrasound probe relative to the target region. However, imaging requires additional equipment and makes the procedure more costly and, in some cases, lack of access to the required imaging equipment can delay or prevent access to treatment. Furthermore, maintaining the position of the ablation device improves the treatment delivery accuracy.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages, and novel features of the disclosure will be set forth in the following detailed description of the disclosure when considered in conjunction with the drawings, which are intended to illustrate, not limit, the invention.

An aspect of the invention is directed to an ultrasound probe comprising a shaft having a proximal end, a tip, and a length measured between the proximal end and the tip with respect to an axis; a plurality of channels defined in the shaft and extending from the proximal end of the shaft along at least a portion of the length of the shaft, the plurality of channels including an ultrasound channel and a balloon channel; one or more ultrasound transducers disposed in the ultrasound channel; and an offset balloon attached to a distal portion of the shaft and disposed at a predetermined distance from the ultrasound transducer(s), the offset balloon fluidly coupled to the balloon channel to receive or withdraw a fluid to adjust an inflation state of the offset balloon, the offset balloon having a deflated state and an inflated state, wherein in the inflated state the offset balloon has an inflated size that is larger than a target anatomical opening such that an inflated offset balloon is configured to mechanically engage the target anatomical opening when the shaft is retracted with respect to the target anatomical opening, and the ultrasound transducer(s) is/are aligned with respect to a target volume when the inflated offset balloon mechanically engages the target anatomical opening.

In one or more embodiments, the ultrasound probe further comprises a coating on the offset balloon. In one or more embodiments, the coating comprises a medication, an antibacterial agent, and/or a lubricant.

In one or more embodiments, the offset balloon is attached to a distal end of the shaft. In one or more embodiments, the offset balloon is a first offset balloon, the balloon channel is a first balloon channel, the predetermined distance is a first predetermined distance, the plurality of channels includes a second balloon channel, the fluid is a first fluid, and the ultrasound probe further comprises a second offset balloon attached to the distal portion of the shaft and disposed at a second predetermined distance from the ultrasound transducer(s), the second offset balloon fluidly coupled to the second balloon channel to receive or withdraw a second fluid to adjust the inflation state of the second offset balloon, the second offset balloon having the deflated state and the inflated state, the second balloon in the inflated state having a size that is larger than the target anatomical opening, whereby an alignment with respect to the target volume can be adjusted by transitioning the first offset balloon to the inflated state or by transitioning the second offset balloon to the inflated state.

In one or more embodiments, the ultrasound probe further comprises a handle attached to the proximal end of the shaft; and a fluid-collection ring disposed on a distal side of the handle, the fluid-collection ring having an inner edge and an outer edge, the inner edge defining a hole through which the shaft extends, a distal side of the fluid-collection ring having a concave shape and configured to direct a liquid away from the handle. In one or more embodiments, the inner edge and the shaft define a fluid-collection channel and the medical device further comprises a fluid-collection reservoir that is fluidly coupled to the fluid-collection channel. In one or more embodiments, a drainage hole is defined through a proximal side and the distal side of the fluid-collection ring and a drainage tube is fluidly coupled to the drainage hole on the proximal side of the fluid-collection ring. In one or more embodiments, the axis is a first axis, the fluid-collection ring is elongated with respect to a second axis compared to with respect to a third axis, and the first, second, and third axes are mutually orthogonal.

In one or more embodiments, the ultrasound probe further comprises a handle attached to the proximal end of the shaft; and a fluid-collection ring disposed on a distal side of the handle, the fluid-collection ring having an inner edge, an outer edge, and a fluid-collection channel defined on a distal side of the fluid-collection ring. In one or more embodiments, the fluid-collection channel is defined only on a distal surface of the fluid-collection ring. In one or more embodiments, the fluid-collection channel extends to a hole defined in the outer edge of the fluid-collection ring. In one or more embodiments, the hole is oriented at a bottom of the fluid-collection channel when the shaft is oriented parallel to a horizontal axis.

Another aspect of the invention is directed to an ultrasound probe comprising a shaft having a proximal end, a tip, and a length measured between the proximal end and the tip with respect to an axis; a plurality of channels defined in the shaft and extending from the proximal end of the shaft along at least a portion of the length of the shaft, the plurality of channels including an ultrasound channel and a balloon channel; one or more ultrasound transducers disposed in the ultrasound channel; and an offset balloon attached to a middle portion of the shaft and disposed at a predetermined distance from the ultrasound transducer(s), the offset balloon fluidly coupled to the balloon channel to receive or withdraw a fluid to adjust an inflation state of the offset balloon, the offset balloon having a deflated state and an inflated state, wherein in the inflated state the offset balloon has an inflated size that is larger than a target anatomical opening such that an inflated offset balloon is configured to mechanically engage the target anatomical opening when the shaft is inserted into the target anatomical opening, and the ultrasound transducer(s) is/are aligned with respect to a target volume when the inflated offset balloon mechanically engages the target anatomical opening.

In one or more embodiments, the ultrasound probe further comprises a coating on the offset balloon. In one or more embodiments, the coating comprises a medication, an antibacterial agent, and/or a lubricant.

In one or more embodiments, the offset balloon is a first offset balloon, the balloon channel is a first balloon channel, the predetermined distance is a first predetermined distance, the plurality of channels includes a second balloon channel, the fluid is a first fluid, and the ultrasound probe further comprises a second offset balloon attached to a distal portion of the shaft and disposed at a second predetermined distance from the ultrasound transducer(s), the second offset balloon fluidly coupled to the second balloon channel to receive or withdraw a second fluid to adjust the inflation state of the second offset balloon, the second offset balloon having the deflated state and the inflated state.

In one or more embodiments, the ultrasound probe further comprises a handle attached to the proximal end of the shaft; and a fluid-collection ring disposed on a distal side of the handle, the fluid-collection ring having an inner edge and an outer edge, the inner edge defining a hole through which the shaft extends, a distal side of the fluid-collection ring having a concave shape and configured to direct a liquid away from the handle.

Another aspect of the invention is directed to a method for performing thermal therapy, comprising inserting a distal portion of a shaft of an ultrasound probe into a target anatomical opening, the shaft having a proximal end, a tip, and a length measured between the proximal end and the tip with respect to an axis, the ultrasound probe further comprising a plurality of channels defined in the shaft and extending from the proximal end of the shaft along at least a portion of the length of the shaft, the plurality of channels including an ultrasound channel and a balloon channel; one or more ultrasound transducers disposed in the ultrasound channel; and an offset balloon attached to the distal portion of the shaft, the offset balloon fluidly coupled to the balloon channel to receive or withdraw a fluid to adjust an inflation state of the offset balloon, the offset balloon in a deflated state. The method further comprises inflating the offset balloon while the distal portion of the shaft, including the offset balloon, is disposed in the target anatomical structure; retracting the shaft until the offset balloon mechanically engages the target anatomical opening, the offset balloon in an inflated state and having an inflated size that is larger than the target anatomical opening, the ultrasound transducer(s) disposed at a predetermined distance from the offset balloon; aligning the ultrasound transducer(s) with respect to a target volume; and applying ultrasound energy, with the ultrasound transducers, to the target volume while the ultrasound transducers are aligned with the target volume.

In one or more embodiments, the offset balloon is a first offset balloon, the balloon channel is a first balloon channel, the predetermined distance is a first predetermined distance, the plurality of channels includes a second balloon channel, the fluid is a first fluid, and the method further comprises after retracting the shaft until the first offset balloon mechanically engages the target anatomical opening, determining that the ultrasound transducer(s) is/are not aligned with respect to the target volume; deflating the first offset balloon; inflating a second offset balloon attached to the distal portion of the shaft, the second offset balloon fluidly coupled to the second balloon channel to receive or withdraw a second fluid to adjust the inflation state of the second offset balloon; and retracting the shaft until the second offset balloon mechanically engages the target anatomical opening, the second offset balloon in the inflated state and having an inflated size that is larger than the target anatomical opening, the ultrasound transducer(s) disposed at a second predetermined distance from the second offset balloon, wherein the ultrasound transducer(s) is/are aligned with respect to the target volume at the second predetermined distance from the second offset balloon.

### BRIEF DESCRIPTION OF THE DRAWINGS

For a fuller understanding of the nature and advantages of the concepts disclosed herein, reference is made to the detailed description of preferred embodiments and the accompanying drawings.

FIG. 1 is a block diagram of a medical system according to one or more embodiments.

FIG. 2A is a top view and partially transparent view of an ultrasound applicator according to one or more embodiments with a balloon in a deflated state.

FIG. 2B is a top view and partially transparent view of the ultrasound applicator shown in FIG. 2A with the balloon in an inflated state.

FIGS. 3A and 3B are end views of the ultrasound applicator shown in FIGS. 2A and 2B, respectively, with the balloon in a deflated state and in an inflated state, respectively.

FIG. 4 is a flow chart of a method for performing thermal therapy using an ultrasound applicator according to one or more embodiments.

FIGS. 5-7 show different steps of the flow chart illustrated in FIG. 4.

FIG. 8A is a top view and partially transparent view of an ultrasound applicator according to one or more embodiments with the balloons in a deflated state.

FIG. 8B is a top view and partially transparent view of the ultrasound applicator shown in FIG. 8A with the balloons in an inflated state.

FIG. 9 is a flow chart of a method for performing thermal therapy using an ultrasound applicator according to one or more embodiments.

FIG. 10 shows a step of the flow chart illustrated in FIG. 9.

FIG. 11 shows an example of an ultrasound applicator having a first balloon in an inflated state and a second balloon in a deflated state.

FIG. 12 shows another step of the flow chart illustrated in FIG. 9.

FIG. 13 shows an example of an ultrasound applicator having a first balloon in a deflated state and a second balloon in an inflated state.

FIG. 14 shows another step of the flow chart illustrated in FIG. 9.

FIG. 15 is a top view and partially transparent view of an ultrasound applicator according to one or more embodiments with a balloon disposed on a middle region of the shaft.

FIG. 16 is a simplified and partially transparent side view of a medical apparatus according to one or more embodiments.

FIG. 17A is a simplified and partially transparent side view of a medical apparatus according to one or more embodiments.

FIG. 17B and 17C are cross sections of the medical apparatus illustrated in FIG. 17A according to one or more embodiments.

FIG. 18 is a simplified and partially transparent side view of a medical apparatus according to one or more embodiments.

FIG. 19A is an isometric view of an ultrasound applicator according to one or more embodiments.

FIG. 19B is an enlarged view of a portion of the ultrasound applicator illustrated in FIG. 19A according to one or more embodiments.

FIGS. 20A and 20B are isolated isometric views of the fluid-collection ring, shown in FIGS. 19A and 19B, from different perspectives.

FIG. 21A is an enlarged view of a portion of the ultrasound applicator and a fluid-collection ring according to one or more embodiments.

FIG. 21B is an isolated isometric view of the fluid-collection ring shown in FIG. 21A according to one or more embodiments.

FIG. 22A is an enlarged view of a portion of the ultrasound applicator and a fluid-collection ring according to one or more embodiments.

FIG. 22B is an isolated isometric view of the fluid-collection ring shown in FIG. 22A according to one or more embodiments.

FIG. 23A is an enlarged view of a portion of the ultrasound applicator and a fluid-collection ring according to one or more embodiments.

FIG. 23B is an isolated isometric view of the fluid-collection ring shown in FIG. 23A according to one or more embodiments.

FIG. 24A is an enlarged view of a portion of the ultrasound applicator and a fluid-collection ring according to one or more embodiments.

FIG. 24B is an isolated isometric view of the fluid-collection ring shown in FIG. 24A according to one or more embodiments.

FIG. 25A is an enlarged view of a portion of the ultrasound applicator and a fluid-collection ring according to one or more embodiments.

FIG. 25B is an isolated isometric view of the fluid-collection ring shown in FIG. 25A according to one or more embodiments.

### DETAILED DESCRIPTION

An ultrasound therapy probe includes one or more balloon(s) attached to a distal portion and/or a middle portion of a shaft of the ultrasound probe. The distance between the balloon(s) and the ultrasound transducer(s) is predetermined and can be used to align the ultrasound transducer(s) with respect to a target volume without using imaging. For example, a distal portion of the ultrasound probe can be inserted into an opening of an internal body cavity or organ, such as the bladder, with a balloon in the deflated state. The balloon can then be inflated while the distal portion of the ultrasound probe remains in the internal body cavity or organ. When the ultrasound probe is retracted, the inflated balloon physically contacts and/or obstructs the opening of the internal body cavity. At this position, the relative position of the ultrasound transducer(s) with respect to the internal body cavity or organ can be determined without imaging since the balloon is at a known position in the internal body cavity or organ and the relative position of the balloon with respect to the ultrasound transducer(s) is known. In some embodiments, the distance between the balloon and the ultrasound transducer(s) is configured such that when the balloon physically contacts the opening of internal body cavity, the ultrasound transducer(s) is/are aligned with the target volume.

One or more additional balloons can be attached to different axial locations of the shaft to provide different alignment options and distances between each balloon and the ultrasound transducers, for example to accommodate anatomical variations.

A fluid-collection ring can be disposed on a distal end of a handle that is attached to a proximal end of the shaft. The fluid-collection ring has a hole through which the shaft extends. The fluid-collection ring is configured to collect and/or deflect liquids, such as bodily fluids, that may discharge from a patient during a medical procedure. The fluid-collection ring can alternately be referred to as a fluid-deflection ring.

The ultrasound applicator can be coupled to a cooling-fluid reservoir, a syringe or reservoir that stores fluid for the balloon(s), a power source, and/or a controller to form a medical apparatus.

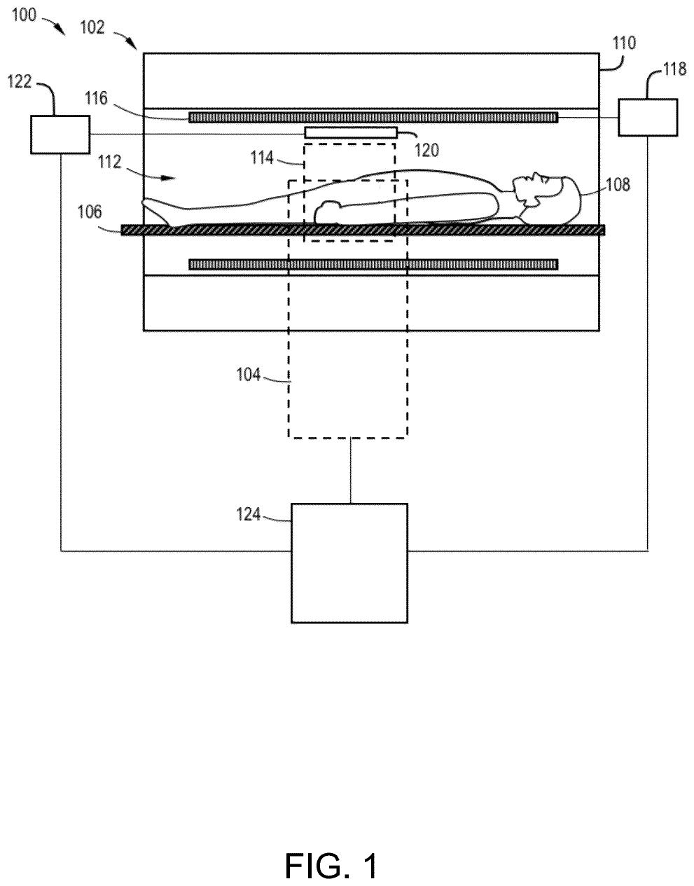

FIG. 1 is a diagram of a medical system 100 in which at least some of the apparatus, systems, and/or methods disclosed herein are employed, in accordance with at least some embodiments. The system 100 includes a patient support 106 (on which a patient 108 is shown), a magnetic resonance imaging (MRI) system 102 and an image-guided energy delivery system 104.

The magnetic resonance system 102 includes a magnet 110 disposed about an opening 112, an imaging zone 114 in which the magnetic field is strong and uniform enough to perform MRI, a set of magnetic field gradient coils 116 to change the magnetic field rapidly to enable the spatial coding of MRI signals, a magnetic field gradient coil power supply 118 that supplies current to the magnetic field gradient coils 116 and is controlled as a function of time, a transmit/receive coil 120 (also known as a “body” coil) to manipulate the orientations of magnetic spins within the imaging zone 114, a radio frequency transceiver 122 connected to the transmit/receive coil 120, and a computer 124, which performs tasks (by executing instructions and/or otherwise) to facilitate operation of the MRI system 102 and is coupled to the radio frequency transceiver 122, the magnetic field gradient coil power supply 118, and the image-guided energy delivery system 104. The image-guided energy delivery system 104 includes a therapeutic applicator, such as an ultrasound applicator, to perform image-guided therapy (e.g., thermal therapy) to treat a treatment volume in the patient 108.

The MRI computer 124 can include more than one computer in some embodiments, at least one of which can be dedicated to the MRI system 102. In at least some embodiments, the MRI computer 124 and/or one or more other computing devices (not shown) in and/or coupled to the system 100 may also perform one or more tasks (by executing instructions and/or otherwise) such as to control the driving or operating frequency of the ultrasound elements in the therapeutic applicator, such as at the center frequency (f₀) and/or at a higher harmonic (3f₀) of the center frequency.

One or more of the computers, including computer 124, can include a treatment plan for and/or program instructions for determining a treatment plan (e.g., in real time) for the patient 108 that includes the target treatment volume and the desired or minimal energy (e.g., thermal) dose for the target treatment volume. The treatment plan can also include the desired operating or driving frequency of the ultrasound elements, such as f₀ and/or 3f₀. The computer(s) can use images from the MRI system 102 to image guide the rotational position and insertion-retraction position of the therapeutic applicator. In some embodiments, one or more dedicated computers control the image-guided energy delivery system 104. Some or all of the foregoing computers can be in communication with one another (e.g., over a local area network, a wide area network, a cellular network, a WiFi network, or other network), for example through a software-controlled link to a communication network.

In some embodiments, the treatment plan includes a set of initial parameters for driving each ultrasound element such as its initial frequency, initial phase, and initial amplitude. These parameters can be updated in real time based on the measured temperature of the target volume, for example as determined by MR thermometry.

In other embodiments, the image-guided energy delivery system 104 can be guided with another imaging device, such as an ultrasound imaging device. In other embodiments, the image-guided energy delivery system 104 can be used without an imaging device in which case the image-guided energy delivery system 104 is an energy delivery system 104

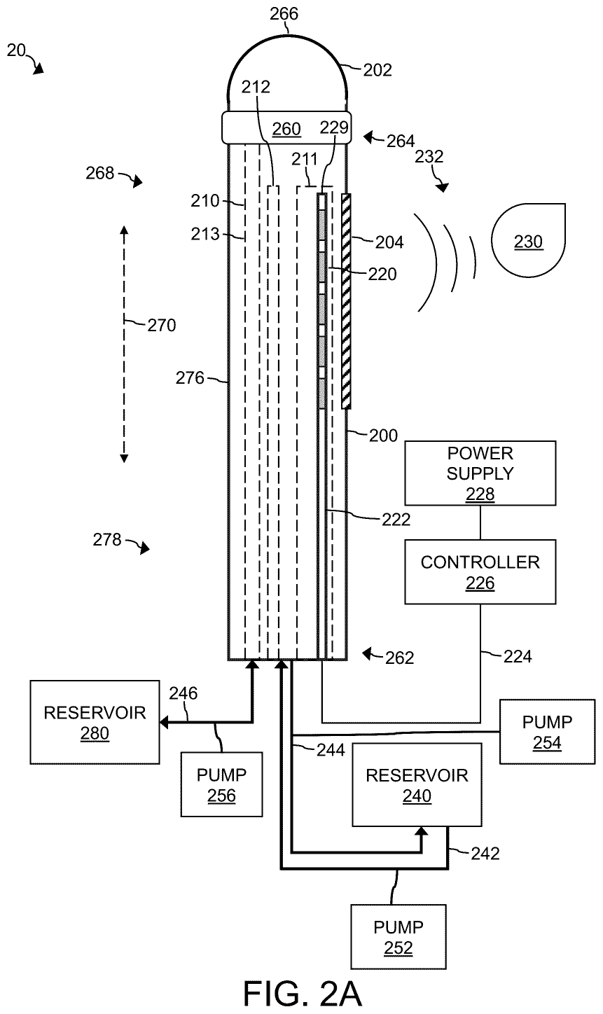

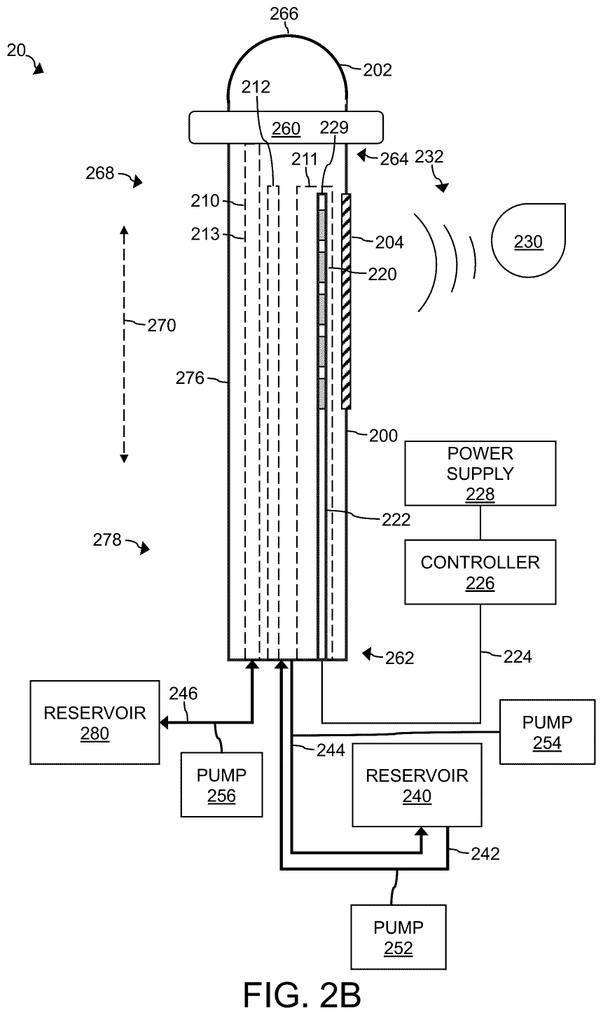

FIG. 2A is a top view and partially transparent view of an ultrasound applicator 20 according to one or more embodiments. The ultrasound applicator 20 can be a therapeutic applicator for an image-guided energy delivery system 104 or an energy delivery system 104 (FIG. 1). The ultrasound applicator 20 includes a shaft 200 attached to or including a tip 202. Multiple channels 210 can be defined in the ultrasound applicator 20. Each channel 210 extends from a proximal end 262 towards or to a distal end 264 of the shaft 200. The shaft 200 and each channel 210 extend parallel to an axis 270, such that the respective lengths of the shaft 200 and each channel 210 can be measured with respect to the axis 270.

The channel(s) 210 can include an ultrasound channel 211 that is configured to receive one or more ultrasound transducers 220. The ultrasound transducer(s) 220 can comprise an array of ultrasound transducers, such as a linear array or a focused array of ultrasound transducers. The ultrasound transducer(s) 220 can be mounted on and/or electrically connected to an elongated circuit board 222. The elongated circuit board 222 can be electrically coupled (e.g., via wire(s) or cable(s) 224) to a controller 226 that can selectively provide electrical power, produced by a power supply 228, at a frequency, relative phase, and/or amplitude according to a treatment plan so as to treat a target volume 230 in a patient. The controller 226 and the power supply 228 can be combined in some embodiments. Ultrasound energy 232 produced by the ultrasound transducer(s) 220 can pass through an ultrasound window 204 in the shaft 200 and can be focused, geometrically and/or electronically, onto the target volume 230.

The channel(s) 210 can include a cooling channel 212 that is configured to receive cooling fluid (e.g., a cooling liquid such as water) that can be used to cool the ultrasound applicator 20 and/or the surrounding volume (e.g., surrounding tissue) during ultrasonic treatment. The cooling fluid can be provided from a cooling fluid reservoir 240. The cooling fluid can be recirculated between the cooling fluid reservoir 240 and the cooling channel 212. Cooler (e.g., room temperature) cooling fluid can flow from the cooling fluid reservoir 240 to the cooling channel 212 through an inlet line 242. After passing through at least a portion of the cooling channel 212 and receiving heat from the ultrasound applicator 20 and/or the surrounding volume, warmer cooling fluid can flow from cooling channel 212 to the cooling fluid reservoir 240 through an outlet line 244. A pump 252 can be fluidly coupled to the inlet line 242 and/or a pump 254 can be fluidly coupled to the outlet line 244. Alternatively, pump 252 or 254 can be fluidly coupled to the inlet line 242 and to the outlet line 244.

A balloon 260 is attached to a distal portion 268 of the shaft 200. The distal portion 268 is located further from the proximal end 262 than a proximal portion 278 of the shaft 200. A middle 276 of the shaft 200 is located between the distal and proximal portions 268, 278. In some embodiments, the balloon 260 can be attached at or near (e.g., within about 5 cm of) a distal end 264 of the shaft 200.

The balloon 260 can be attached to the shaft 200 using an adhesive, crosslinking, laser welds, and/or another method. The balloon 260 can be located between the ultrasound transducer(s) 220 and the tip 202, for example between the ultrasound transducer(s) 220 and a distal end 266 of the ultrasound applicator 20. The balloon 260 is located at a predetermined distance from a distal end 229 of the elongated circuit board 222 and/or from one or more of the ultrasound transducer(s) 220. The distance can be measured with respect to the axis 270. The balloon 260 can alternately be referred to as an offset balloon. In some embodiments, two or more balloons can be attached at or near the distal end 264 of the shaft 200.

The balloon 260 is fluidly coupled to a balloon channel 213 to receive or remove fluid to adjust an inflation state of the balloon 260. The balloon channel 213 can be fluidly coupled to a reservoir 280 that can hold fluid to inflate the balloon 260 and/or to receive fluid from the balloon 260 to deflate the balloon 260. The fluid can flow through a balloon line 246 that can be fluidly coupled to the reservoir 280 and the balloon channel 213. A pump and/or a vacuum 256 can be fluidly coupled to the reservoir 280 and/or the balloon line 246. In some embodiments, the reservoir 280 and optionally the balloon line 246 can be replaced with a syringe that can supply a volume (e.g., a predetermined volume) of fluid (e.g., air or liquid) to inflate the balloon 260 and/or that can receive a volume (e.g., a predetermined volume) of fluid (e.g., air or liquid) to deflate the balloon 260.

The balloon 260 is a deflated state in FIG. 2A and is in an inflated state in FIG. 2B.

The balloon 260 can be configured and/or sized to mechanically engage an anatomical feature in a mammal, such as a person, such as during a medical procedure. When the balloon 260 mechanically engages an anatomical feature, the distance between the anatomical feature and the ultrasound transducer(s) 220 can be determined without using imaging because the distance between the balloon 260 and the elongated circuit board 222 and/or the ultrasound transducer(s) 220 is known. The distance between the balloon 260 and the elongated circuit board 222 and/or the ultrasound transducer(s) 220 can be configured to align the ultrasound transducer(s) 220 with a target volume 230 when the balloon 260 mechanically engages an anatomical feature.

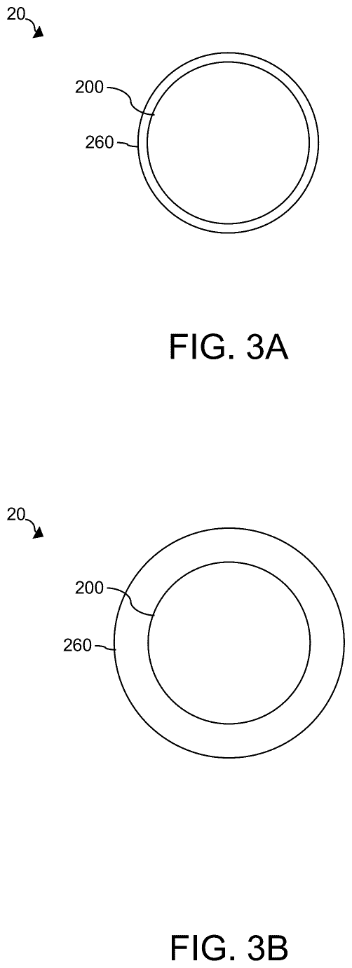

The balloon 260 is disposed and attached about a circumference of shaft 200, for example as illustrated in FIGS. 3A and 3B which are end views of the ultrasound applicator 20 with the balloon 260 in a deflated state and in an inflated state, respectively.

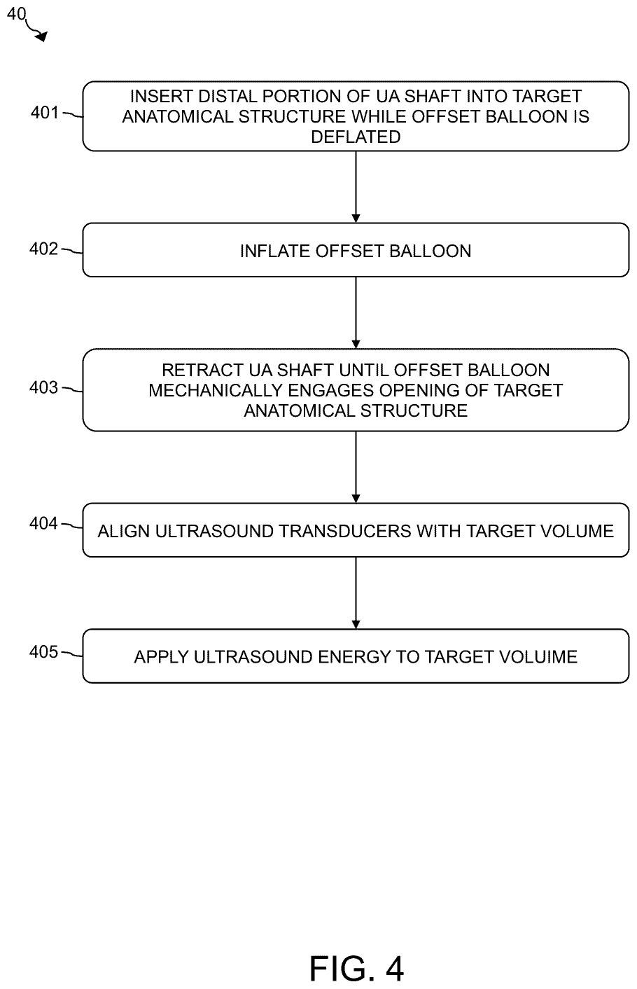

FIG. 4 is a flow chart of a method 40 for performing thermal therapy using an ultrasound applicator 20 according to one or more embodiments.

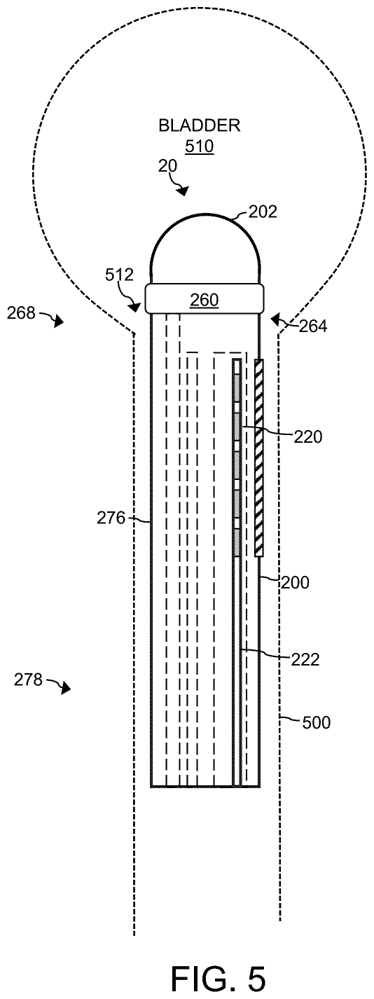

In step 401, a distal portion 268 of a shaft 200 of the ultrasound applicator 20, including a balloon 260, is inserted into a target anatomical structure such as a bladder 510, for example as shown in FIG. 5. The distal portion 268 of the shaft 200 can be inserted into an opening 512 of the bladder 510 through the urethra 500. The middle 276 and/or the proximal portion 278 of the shaft 200 can remain in the urethra 500 when the distal portion 268 of the shaft 200 is inserted into the bladder 510. The balloon 260 is in a deflated state during step 401. A user, such as a doctor or technician, can approximate the distance to insert the ultrasound applicator 20 such that imaging is optionally not used in this step.

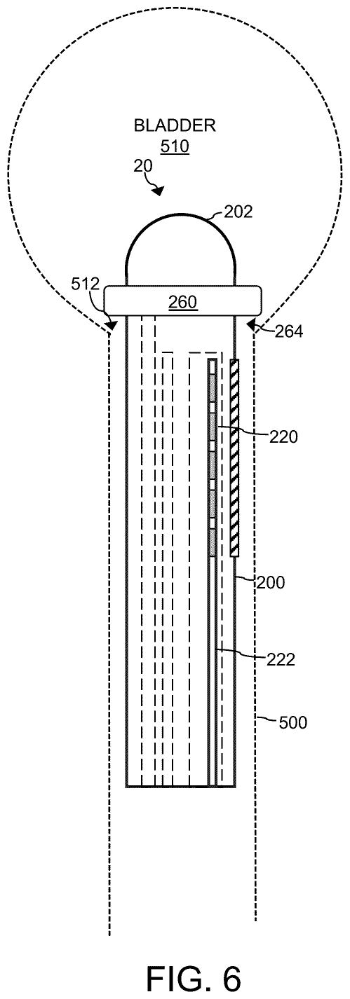

In step 402, the balloon 260 is inflated. The balloon 260 can be inflated by adding fluid to the balloon 260, for example as shown in FIG. 6. For example, saline or another liquid can be added to the balloon 260 from a syringe or a fluid reservoir 280. In another example, a gas such as air can be added to the balloon 260 from a syringe or a pump.

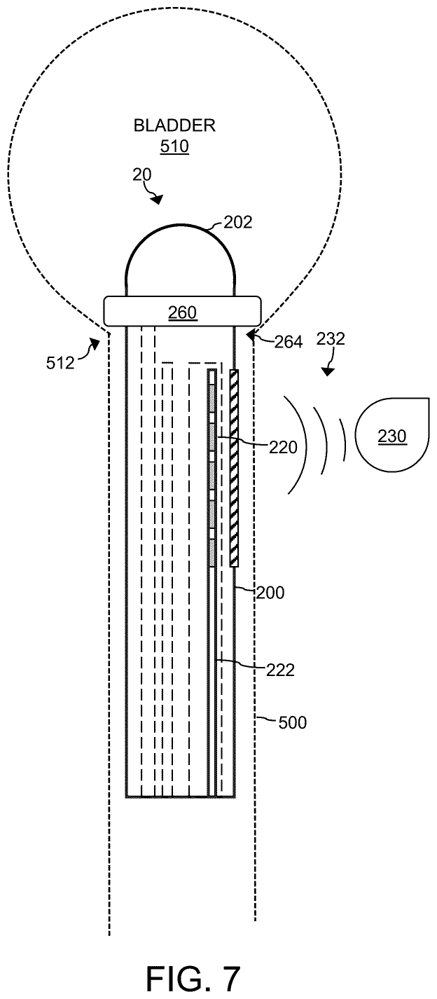

In step 403, the ultrasound applicator 20 (e.g., the shaft 200) is retracted until the inflated balloon 260 mechanically engages the opening 512 of the bladder 510 for example as shown in FIG. 7. The ultrasound applicator 20 is retracted in a proximal direction away from the bladder 510 and towards the urethra 500. When the ultrasound applicator 20 is retracted, a proximal side of the inflated balloon 260 mechanically engages and/or directly physically contacts a proximal end of the bladder 510 at or near a distal side of the opening 512 (e.g., at an interface of the urethra 500 and bladder 510). The balloon 260 is configured to have an inflated diameter (or another measurement such as width) that is larger than the internal diameter of the urethra 500 such that, after the balloon 260 is inflated, the balloon 260 blocks or obstructs the ultrasound applicator 20 from being retracted through the opening 512 and into the urethra 500.

In step 404, the ultrasound transducer(s) 220 is/are aligned with a target volume 230. Since the distance between the balloon 260 and the ultrasound transducer(s) 220 is known and the balloon 260 is in a known location at or near a distal side of the opening 512 of the bladder 510, the distance between the ultrasound transducer(s) 220 and the opening 512 can be determined without imaging. Using this information, the ultrasound transducer(s) 220 is positioned and/or aligned to treat a target volume 230, such as a region of the prostate, without imaging.

After the ultrasound transducer(s) 220 is/are aligned with the target volume 230, ultrasound energy 232 is applied to the target volume 230 by the ultrasound transducer(s) 220 in step 405.

The example medical procedure is described with respect to the urethra 500 and bladder 510. In other examples, the ultrasound applicator 20 can be used to perform medical procedures with respect to other anatomical features.

In one or more embodiments, an ultrasound applicator can include multiple balloons. The balloons can be placed at different distances from the ultrasound transducer(s) and/or from the distal end of the shaft to provide different options to position the ultrasound transducer(s) relative to a target volume.

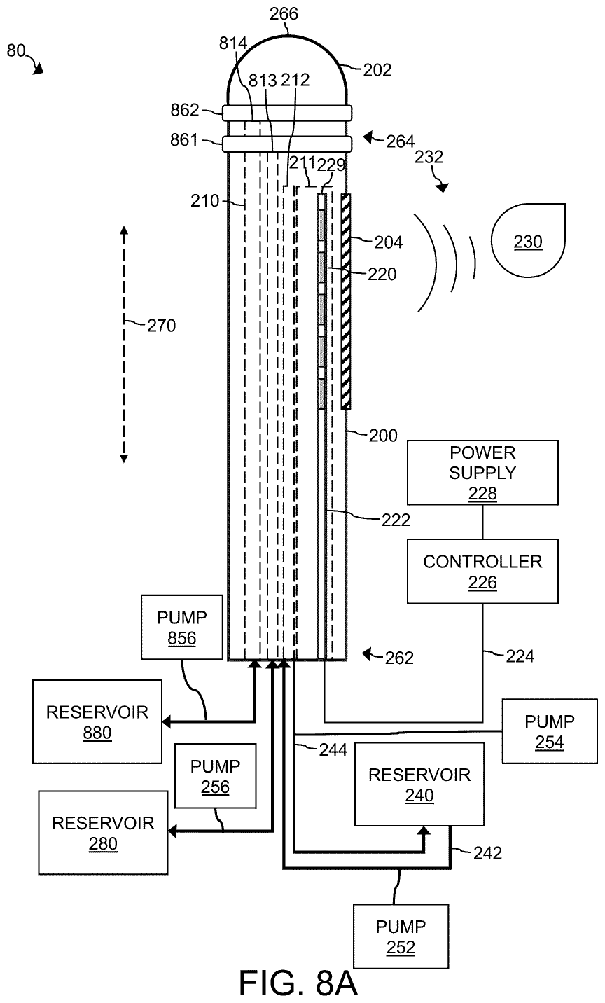

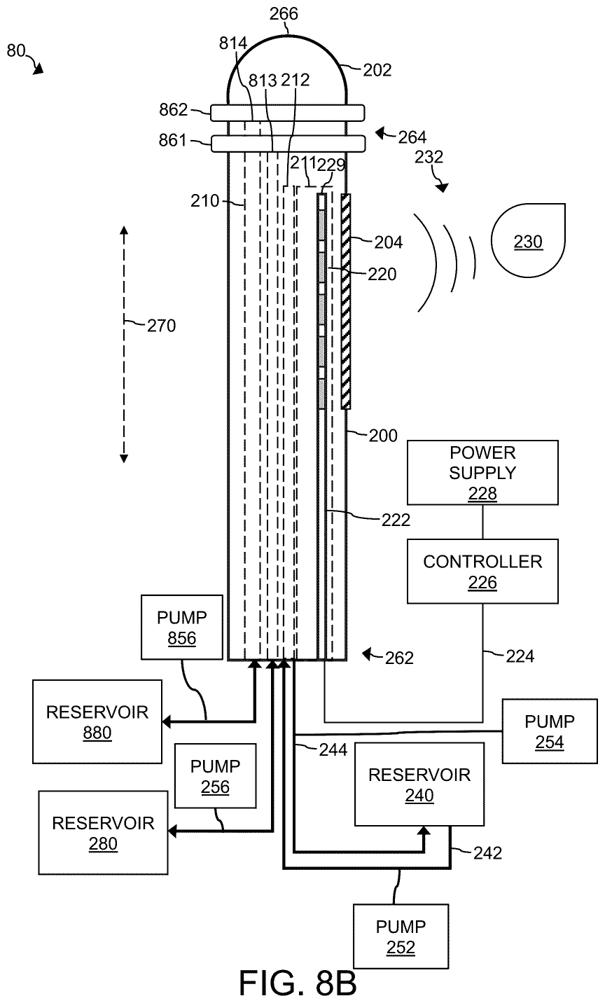

FIG. 8A is a top view and partially transparent view of an ultrasound applicator 80 according to one or more embodiments. The ultrasound applicator 80 is the same as the ultrasound applicator 20 except that the ultrasound applicator 80 includes include a plurality of balloons 861, 862 and a respective plurality of balloon channels 813, 814. The ultrasound applicator 80 can include additional balloons and/or additional balloon channels in one or more embodiments.

Each balloon 861, 862 is located at a respective predetermined distance from the distal end 229 of the elongated circuit board 222, from the ultrasound transducer(s) 220, and/or from the distal end 266 of the ultrasound applicator 20. Each balloon 861, 862 can be the same as or different than balloon 260 and can be referred to as a respective balloon. The first balloon 861 is fluidly coupled to a first balloon channel 813, and the second balloon 862 is fluidly coupled to a second balloon channel 814. Fluid can be provided to and/or received from each balloon 861, 862 through the respective balloon channel 813, 814 to change the respective inflation state of each balloon 861, 862. Thus, each balloon 861, 862 can be inflated and/or deflated independently. The first and second balloon channels 813, 814 can be fluidly coupled to respective reservoirs or syringes in the same manner as discussed above with respect to the balloon channel 213. For example, the first and second balloons 861, 862 can be fluidly coupled to respective reservoirs 280, 880 through the respective first and second balloon channels 813, 814 (e.g., using respective pumps 256, 856) to receive or provide fluid to inflate or deflate one or both balloons 861, 862. Alternatively, one or both balloons 861, 862 can be fluidly coupled to a respective syringe through the respective first and second balloon channels 813, 814. A single syringe can also be used to inflate one of the balloons 861, 862 and then the other of the balloons 862, 861.

The first and second balloons 861, 862 are in a deflated state in FIG. 8A and in an inflated state in FIG. 8B.

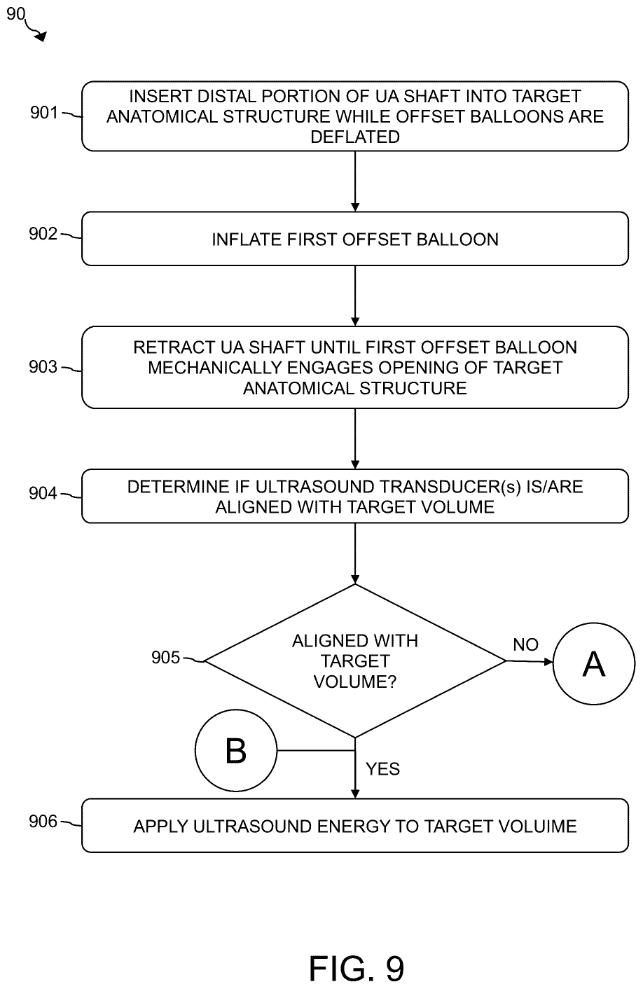

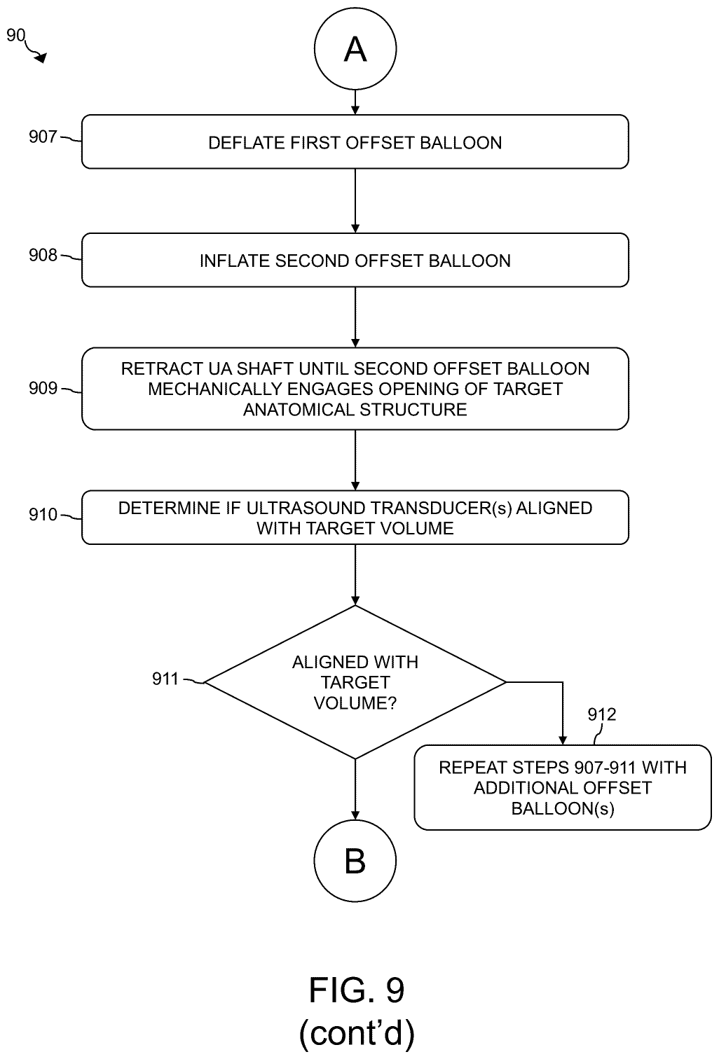

FIG. 9 is a flow chart of a method 90 for performing thermal therapy using an ultrasound applicator 80 according to one or more embodiments.

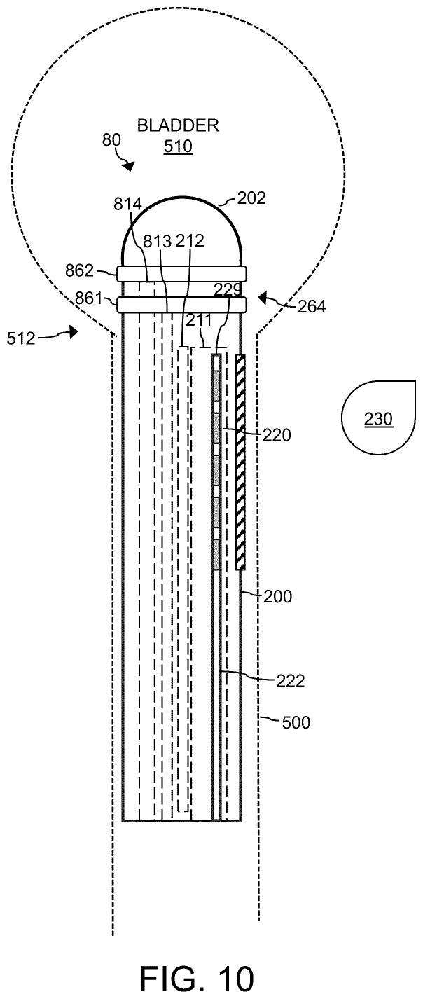

In step 901, a distal portion 268 of a shaft 200 of the ultrasound applicator 80, including at least first and second balloons 861, 862, are inserted into a target anatomical structure such as a bladder 510, for example as shown in FIG. 10. The distal portion 268 of the shaft 200 can be inserted into an opening 512 of the bladder 510 through the urethra 500. The middle 276 and/or the proximal portion 278 of the shaft 200 can remain in the urethra 500 when the distal portion 268 of the shaft 200 is inserted into the bladder 510. The balloons 861, 862 are in a deflated state during step 901. A user, such as a doctor or technician, can approximate the distance to insert the ultrasound applicator 80 such that imaging is optionally not used in this step.

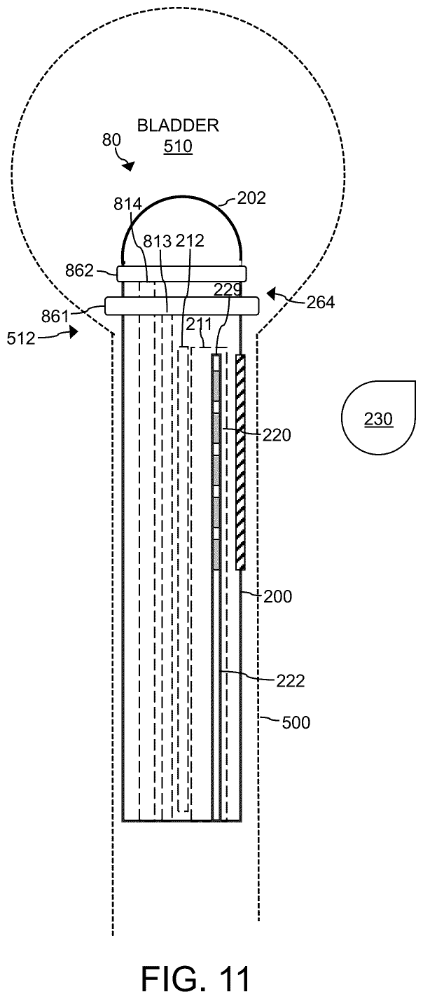

In step 902, a first balloon is inflated. The balloon inflated can be the first balloon 861 or the second balloons 862. The first balloon can be inflated by adding fluid thereto. For example, saline or another liquid can be added to the balloon 861 or 862 from a syringe or a respective fluid reservoir 280, 880. In another example, a gas such as air can be added to the balloon 861 or 862 from a syringe or a pump. An example of the first balloon 861 in an inflated state (while the second balloon 862 is in a deflated state) is shown in FIG. 11.

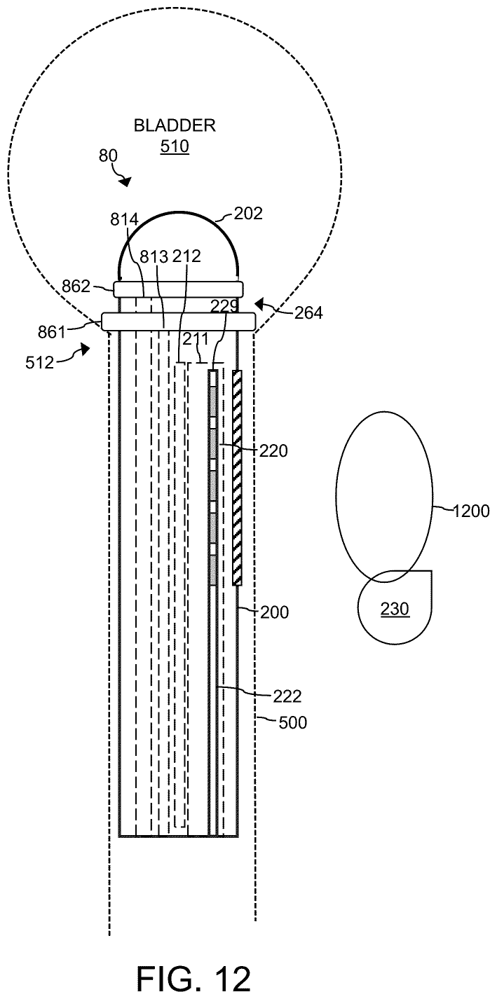

In step 903, the ultrasound applicator 80 (e.g., the shaft 200) is retracted until the inflated balloon 861 (or 862) mechanically engages the opening 512 of the bladder 510 for example as shown in FIG. 12. The ultrasound applicator 20 is retracted in a proximal direction away from the bladder 510 and towards the urethra 500. When the ultrasound applicator 20 is retracted, a proximal side of the inflated balloon 260 mechanically engages and/or directly physically contacts a proximal end of the bladder 510 at or near a distal side of the opening 512 (e.g., at an interface of the urethra 500 and bladder 510). The balloon 260 is configured to have an inflated diameter (or another measurement such as width) that is larger than the internal diameter of the opening 512 of the urethra 500 such that, after the balloon 260 is inflated, the balloon 260 blocks or obstructs the ultrasound applicator 20 from being retracted through the opening 512 and into the urethra 500.

In step 904, it is determined whether the ultrasound transducer(s) 220 is/are aligned with a target volume 230. For example, low intensity ultrasound can be applied by the ultrasound transducer(s) 220 and the temperature of the tissue can be measured (e.g., with MRI thermometry) to determine where the position and orientation of the ultrasound transducer(s) 220. Additionally or alternatively, one or more fiducial marks on the ultrasound applicator 20 can be used to determine the position and/or orientation of the ultrasound transducer(s) 220. The fiducial marks can be detected using imaging such as with an MRI system and/or a imaging ultrasound device/probe. Alignment of the ultrasound transducer(s) 220 with the target volume 230 can determined without imaging in one or more embodiments.

If the ultrasound transducer(s) 220 is/are aligned with the target volume 230 (i.e., step 905=yes), then therapeutic ultrasound energy is applied to the target volume 230 in step 906 (e.g., according to a treatment plan). If the ultrasound transducer(s) 220 is/are not aligned with the target volume 230 (i.e., step 905=no), the method 90 proceeds to step 907 (via placeholder A).

Since the distance between the first balloon 861 and the ultrasound transducer(s) 220 is known and the first balloon 861 is in a known location at the opening 512 of the bladder 510, the distance between the ultrasound transducer(s) 220 and the opening 512 of the bladder 510 can be determined without imaging. Using this information, the ultrasound transducer(s) 220 can be properly positioned at a first location to treat a target volume 230, such as a region of the prostate, without imaging.

Depending on the mammal's anatomy, the target volume 230 may be located outside the electronic and/or geometric focal zone of the ultrasound transducer(s) 220. For example, in FIG. 12, at least a portion of the target volume 230 can be located in a proximal direction of a focal region or zone 1200 of the ultrasound transducer(s) 220. To adjust to the anatomical features of the mammal, the position of the ultrasound applicator 80 and of the corresponding focal region 1100 can be changed by inflating the second balloon 862 instead of the first balloon 861.

In step 907, the first balloon 861 or 862 is deflated. Continuing with the example where the first balloon inflated in step 902 is the first balloon 861, the first balloon 861 is deflated in step 907. The first balloon 861 can be inflated using a syringe or by emptying the fluid in the first balloon 861 into the fluid reservoir 280, for example by applying a vacuum (e.g., using pump 256 or another pump) to the first balloon channel 813.

In step 908, the second balloon 862 or 861 is inflated. Continuing with the example where the first balloon inflated in step 902 is the first balloon 861, the second balloon 862 is inflated in step 908. The second balloon 862 can be inflated using a syringe or by adding fluid from fluid reservoir 880 to the second balloon 862, for example by applying pressure (e.g., using pump 856 or another pump) to the second balloon channel 814.

The ultrasound applicator 80 can be moved in a distal direction (e.g., further into the bladder 510) before the first balloon is deflated in step 907 and/or before the second balloon is inflated in step 908.

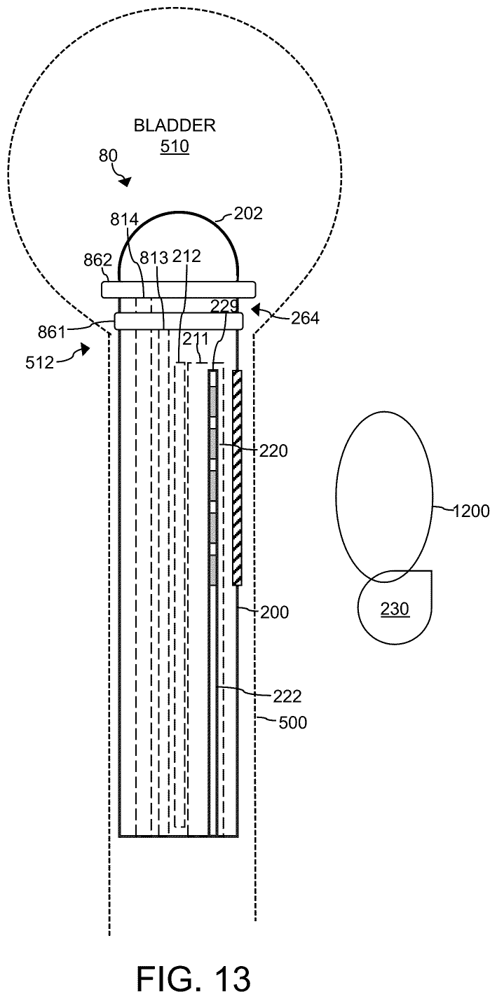

An example of the second balloon 862 in an inflated state (while the first balloon 861 is in a deflated state), following steps 907 and 908, is shown in FIG. 13.

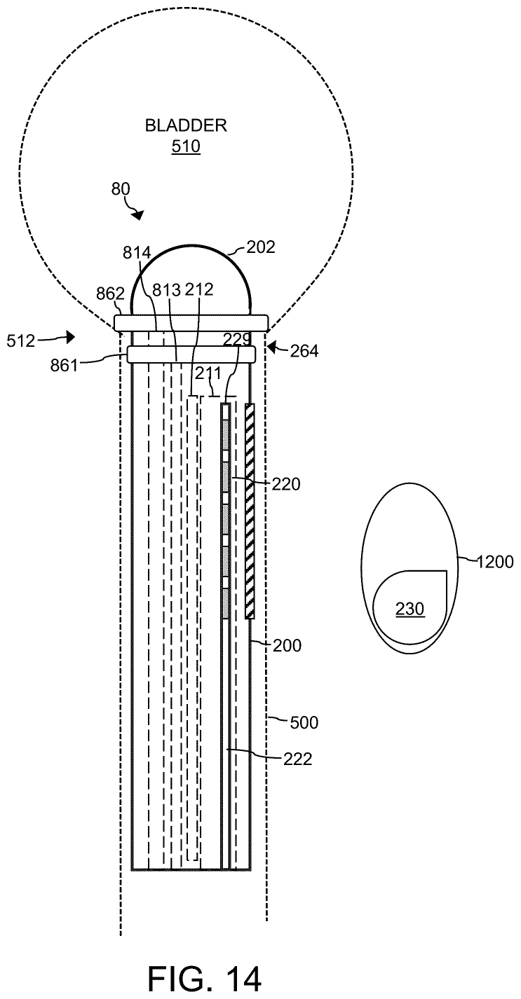

In step 909, the ultrasound applicator 80 (e.g., the shaft 200) is retracted until the inflated second balloon 862 (or 861) mechanically engages the opening 512 of the bladder 510 for example as shown in FIG. 14. The ultrasound applicator 80 is retracted in a proximal direction away from the bladder 510 and towards the urethra 500. When the ultrasound applicator 80 is retracted, a proximal side of the inflated second balloon 862 mechanically engages and/or directly physically contacts a proximal end of the bladder 510 at or near a distal side of the opening 512 (e.g., at an interface of the urethra 500 and bladder 510). The second balloon 862 is configured to have an inflated diameter (or another measurement such as width) that is larger than the internal diameter of the opening 512 of the urethra 500 such that, after the balloon 862 is inflated, the balloon 862 blocks or obstructs the ultrasound applicator 80 from being retracted through the opening 512 and into the urethra 500.

By inflating the second balloon 862 and deflating the first balloon 861, the focal region or zone 1200 is moved in a proximal direction such that the focal region 1100 can overlap the target volume 230, as shown in FIG. 14.

In step 910, it is determined whether the ultrasound transducer(s) 220 is/are aligned with a target volume 230. Step 910 can be performed in the same manner as step 904.

If the ultrasound transducer(s) 220 is/are aligned with the target volume 230 (i.e., step 911=yes), then therapeutic ultrasound energy is applied to the target volume 230 in step 906 (via placeholder B) (e.g., according to a treatment plan). If the ultrasound transducer(s) 220 is/are not aligned with the target volume 230 (i.e., step 911=no), the method 90 proceeds to step 912. In step 912, steps 907-911 are repeated with additional offset balloons. For example, in the next iteration through steps 907-911 the second balloon 862 is deflated in step 907 and a third balloon is inflated in step 908. In step 909, the ultrasound applicator 80 is retracted until the third balloon mechanically engages the opening 512 of the bladder 510.

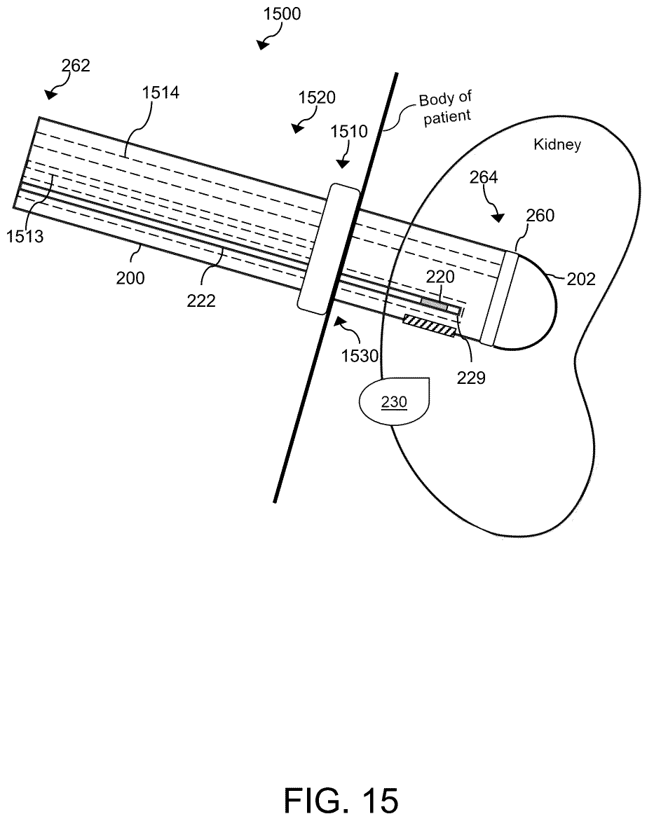

In some embodiments, an ultrasound applicator 1500 can include a balloon 1510 (or multiple balloons) between the proximal and distal ends 262, 264 of the shaft 200, such as in a middle region 1520, as shown in FIG. 15. The balloon 1510 is located at a predetermined distance from the distal end 229 of the elongated circuit board 222 and/or from the ultrasound transducer(s) 220. In the inflated state (as illustrated), the balloon 1510 can mechanically engage an anatomical feature in a mammal, such as a person, for example during a medical procedure. In the illustrated example, the balloon 1510 is inflated to mechanically engage the outside of the body (e.g., skin) of the mammal proximal to an insertion site 1530 (e.g., a surgical or natural opening). When the balloon 1510 mechanically engages an anatomical feature, the distance between the anatomical feature and the ultrasound transducer(s) 220 can be determined without imaging because the distance between the balloon 1510 and the elongated circuit board 222 and/or the ultrasound transducer(s) 220 is known, allowing the ultrasound transducer(s) 220 to be positioned and/or aligned with respect to a target volume 230 without imaging.

The ultrasound applicator 1500 can include one or more additional balloons, such as the balloons 260, 861, and/or 862 described herein. For example, the ultrasound applicator 1500 is illustrated with an optional balloon 260.

The balloon 1510 is fluidly coupled to a first balloon channel 1513, and the optional balloon 260 is fluidly coupled to a second balloon channel 1514. Fluid can be provided to and/or received from each balloon 1510, 260 through the respective balloon channel 1513, 1514 to change the respective inflation state of each balloon 1510, 260. Thus, each balloon 1510, 260 can be inflated and/or deflated independently. The first and second balloon channels 1513, 1514 can be fluidly coupled to respective reservoirs or syringes in the same manner as discussed above with respect to the first and second balloon channels 813, 814.

In some embodiments, one or more of the balloons 260, 861, 862, and/or 1510 can include a coating. The coating can include medication or medicine such as for pain relief, an antibacterial coating (e.g., to reduce the likelihood of bacterial contamination), lubrication, and/or another coating.

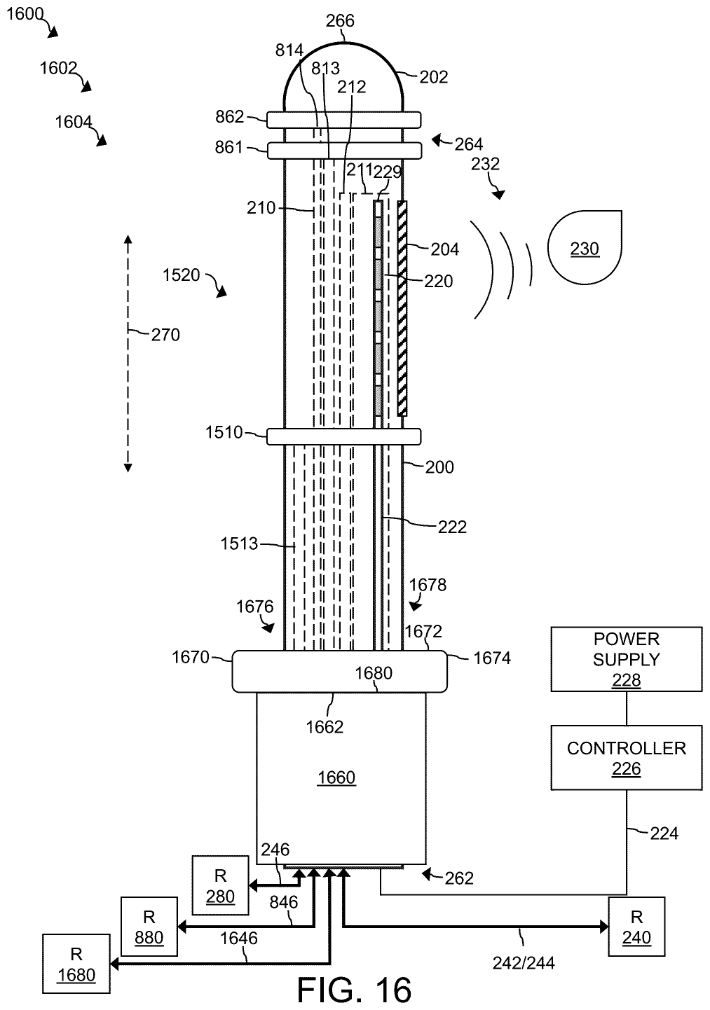

In one or more embodiments, an ultrasound applicator can include one or more balloons (e.g., balloons 260, 861, 862, and/or 1510) and a fluid-collection ring. For example, FIG. 16 shows a medical apparatus 1600 includes a medical device 1602 that includes an ultrasound applicator 1604 and a fluid-collection ring 1670. The ultrasound applicator 1604 includes one or more balloons 861, 862, 1510 and a fluid-collection ring 1670. One, some, or all of the balloons 861, 862, 1510 can be optional in one or more embodiments. The ultrasound applicator 1604 can be the same as the ultrasound applicator 20, 80, or 1500.

The balloon 861 can be fluidly coupled to a reservoir 880 (via a balloon line 846) or a syringe. The balloon 862 can be fluidly coupled to a reservoir 1680 (via a balloon line 1646) or a syringe. The balloon 1510 can be fluidly coupled to a reservoir 280 (via a balloon line 246) or a syringe. The medical apparatus 1600 can include one or more pumps. In one example, each reservoir 240, 280, 880, 1680 is fluidly coupled to a respective pump. The pump(s) are not shown in FIG. 16 to prevent crowding.

The fluid-collection ring 1670 is disposed on a distal side 1662 of a handle 1660 that is attached to the proximal end 262 of the shaft 200. The fluid-collection ring 1670 has an inner edge 1676 that defines a hole 1678 that is configured to receive the shaft 200.

The fluid-collection ring 1670 can be configured to receive and/or collect fluids (e.g., liquids) during use of the ultrasound applicator 1604. Additionally or alternatively, the fluid-collection ring 1670 can be configured to deflect fluids away from the handle 1660 and/or other equipment such as a power supply 228, a controller 226, pump(s) 252, 254, 256, 856, and/or a positioning system (e.g., an automated positioning system) (e.g., shown in FIGS. 8A and 8B but not in FIG. 16 for clarity purposes only).

A distal side 1672 of the fluid-collection ring 1670 can includes a taper, angle, or curve such that, in the orientation illustrated, an outer edge 1674 is higher than the inner edge 1676. In some embodiments, the distal side 1672 has a concave shape, for example that can be formed by a portion of a sphere. The difference in height between the outer edge 1674 and the inner edge 1676 can cause fluids to flow towards the inner edge 1676 and/or away from the handle 1660 and other components of a medical apparatus that includes the ultrasound applicator 1604 such as a power supply, a controller, one or more pump(s), and/or a positioning system.

During a medical procedure, the shaft 200 may be inserted into a natural opening or a surgical opening in a mammalian patient. For example, the shaft 200 may be inserted suprapubicly, transversally, transrectally, transperineally, or transurethrally. When the shaft 200 is inserted into a natural opening such as the urethra, the distal side 1672 of the fluid-collection ring 1670 may physically contact the head of a patient's penis. The distal side 1672 can conform to the head of a penis to improve patient comfort and/or to better collect any fluids discharged (e.g., from the male urethra) during the medical procedure. Additionally or alternatively, the distal side 1672 can conform to another anatomical feature at or near the natural opening or the surgical opening.

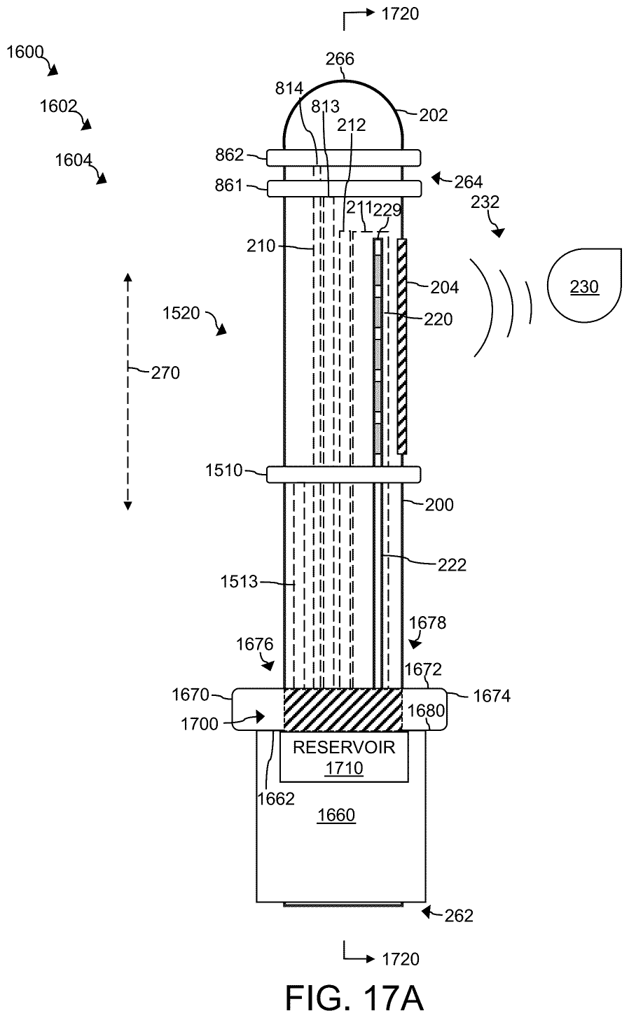

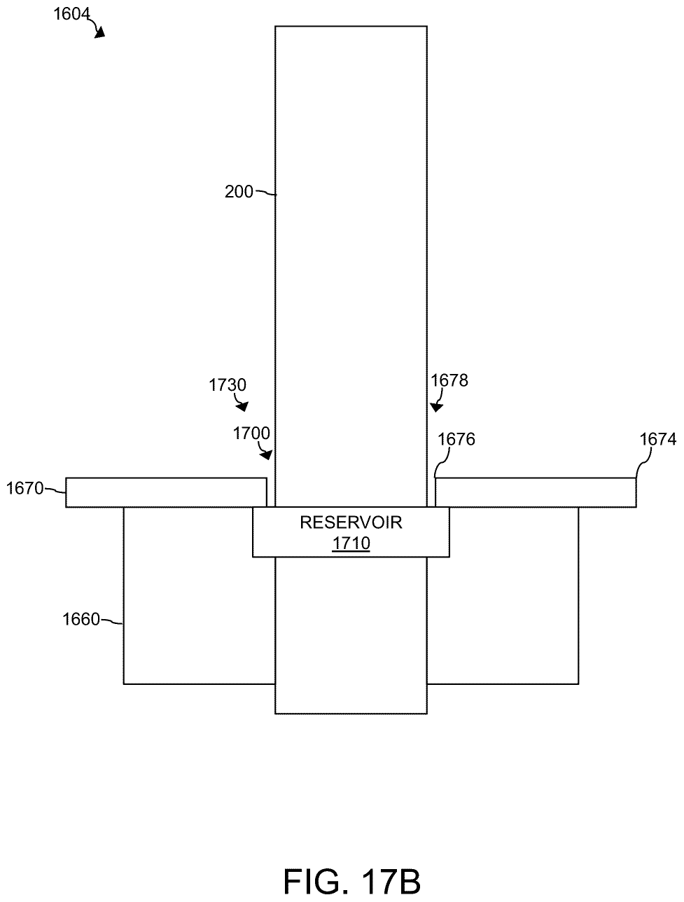

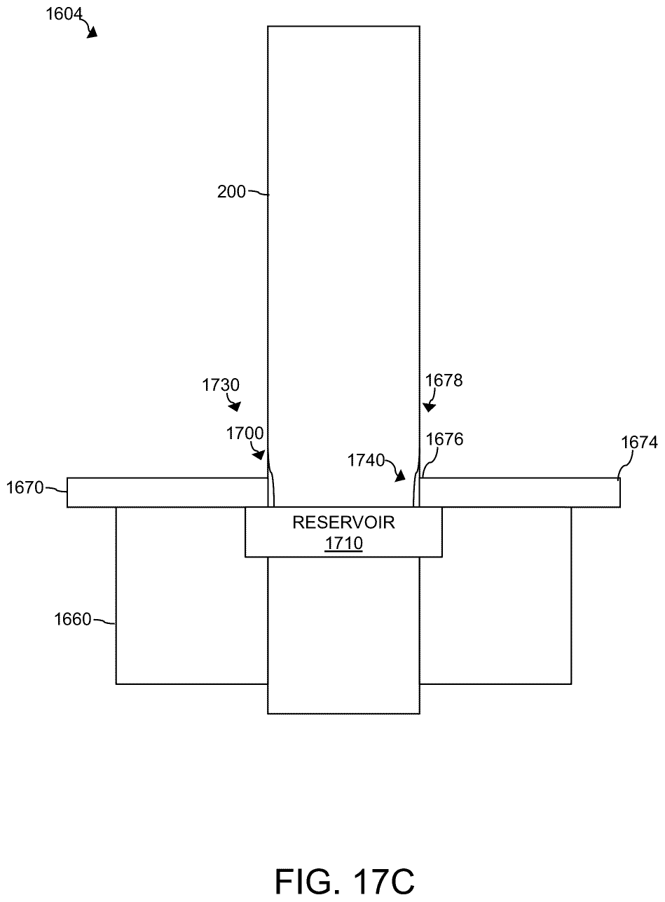

In one or more embodiments, the inner edge 1676 and the shaft 200 form a fluid-tight seal. Any fluid discharged during a medical procedure can be collected and held on the distal side 1672 of the fluid-collection ring 1670 (e.g., depending on the orientation of shaft). In one or more embodiments, a channel 1700 is defined between the inner edge 1676 and the shaft 200, the channel 1700 extending along the outside (e.g., perimeter or circumference) of the shaft 200, as illustrated in FIG. 17A. Any fluid discharged during a medical procedure can be collected on the distal side 1672 of the fluid-collection ring 270 and funneled or directed into the channel 1700. The channel 1700 can be fluidly connected to a fluid-collection reservoir 1710 in the handle 1660. Collecting the discharged fluids prevents them from flowing onto and/or into nearby equipment, such as a power supply, a controller, one or more pump(s), and/or a positioning system, or other equipment, and from flowing onto the floor. The channel 1700 can be formed and/or defined by a gap 1730 between the inner edge 1676 of the fluid-collection ring 1670 and the shaft 200, as shown in FIG. 17B which is a cross section of the ultrasound applicator 1604 taken through plane 1720 in FIG. 3A according to one or more embodiments. Additionally or alternatively, the channel 1700 and/or the gap 1730 can be formed and/or defined by a groove 1740 in the shaft 200 adjacent to the inner edge 1676 of the fluid-collection ring 1670, as shown in FIG. 17C which is a cross section of the ultrasound applicator 1604 taken through plane 1720 in FIG. 17A according to one or more embodiments. The balloons 260, 861, 862, and 1510 are not shown in FIGS. 17B and 17C for clarity purposes only.

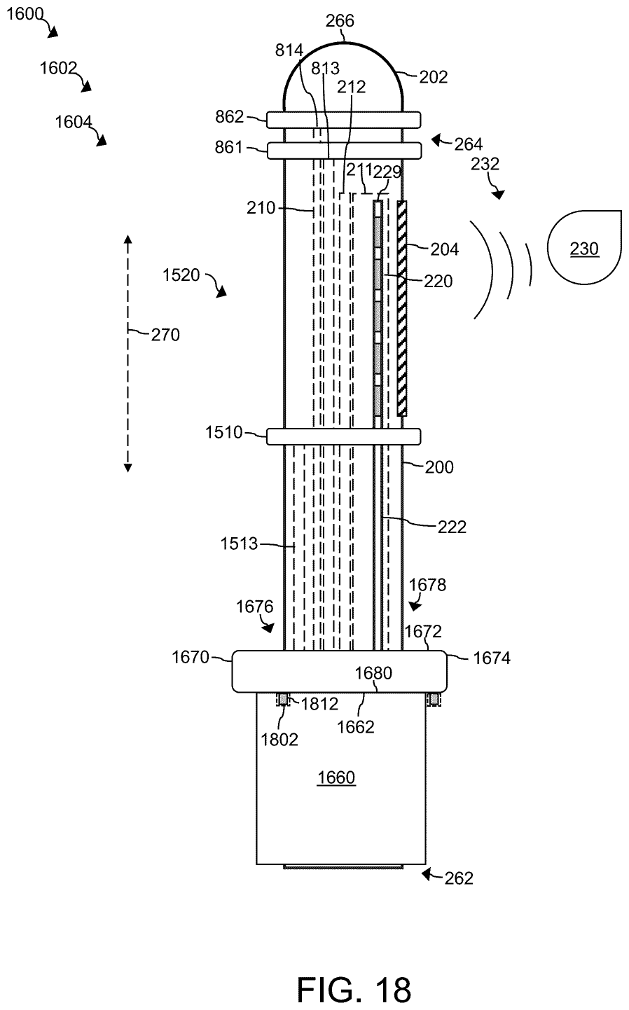

In one or more embodiments, a proximal side 1680 (FIGS. 16, 17A) of the fluid-collection ring 1670 can be attached to the distal side 1662 of the handle 1660 such that the rotational orientation of the fluid-collection ring 1670 remains stationary as the shaft 200 is rotated during therapy. Additionally or alternatively, the distal side 1662 of the handle 1660 can include one or more features 1802 that mechanically engage one or more respective features 1812 on the proximal side 1680 of the fluid-collection ring 1670, as illustrated in FIG. 18, for example keyed and/or complementary features on the distal side 1662 of the handle 1660 and on the proximal side 1680 of the fluid-collection ring 1670. The embodiment illustrated in FIG. 18 can be combined with the embodiments illustrated in FIGS. 16, 17A, 17B, and/or 17C.

The fluid-collection ring 1670 can be removable in some embodiments. For example, depending on the size of the head of the patient's penis (or other anatomical feature to which the fluid-collection ring 1670 can conform), the fluid-collection ring 1670 may be removed and replaced with another fluid-collection ring that has a larger or smaller size that more closely matches the size of the head of the patient's penis (or other anatomical feature to which the fluid-collection ring 1670 can conform).

The fluid-collection ring 1670 can be formed out of a biocompatible material such as a biocompatible polymer or plastic. The fluid-collection ring 1670 can be configured to be placed on other thermal therapy devices instead of the ultrasound applicator 1604.

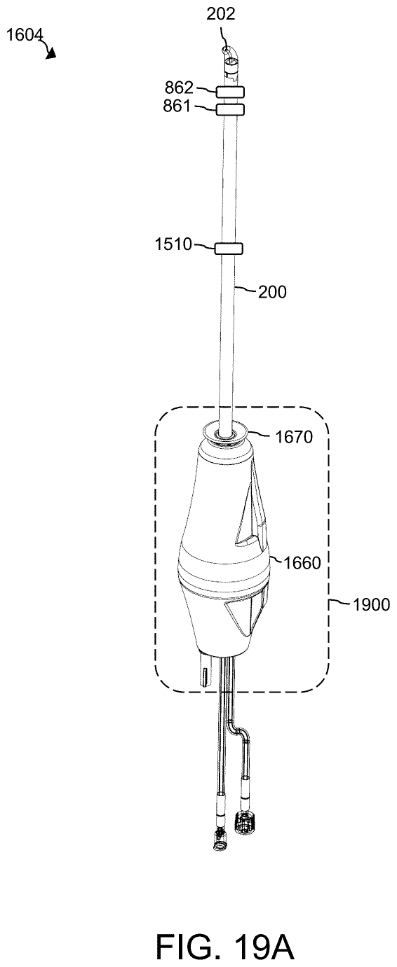

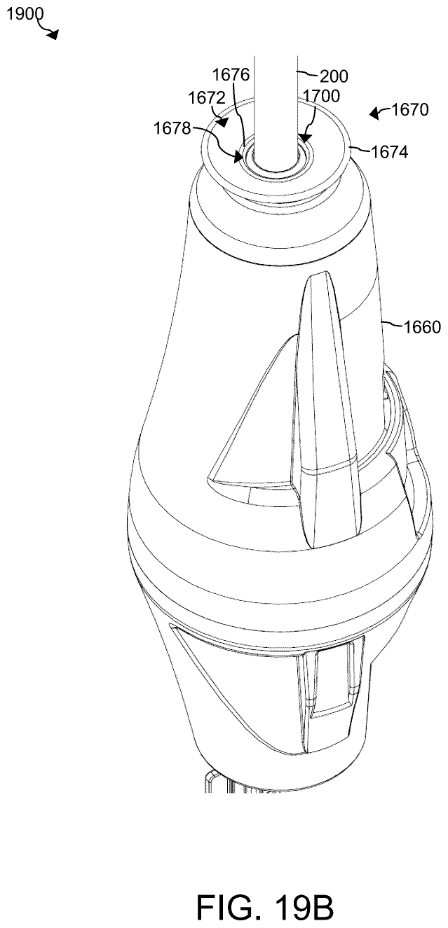

FIG. 19A is an isometric view of the ultrasound applicator 1604 according to one or more embodiments to further illustrate the fluid-collection ring 1670.

FIG. 19B is an enlarged view of region 1900 in FIG. 19A to further illustrate the fluid-collection ring 1970 including inner edge 1676, the channel 1700, and an example concave distal side 1672. The distal side 1672 of the fluid-collection ring 1670 has a circular outer edge 1674. In other embodiments, the outer edge 1674 can define an oval, a hexagon, an octagon, or another shape.

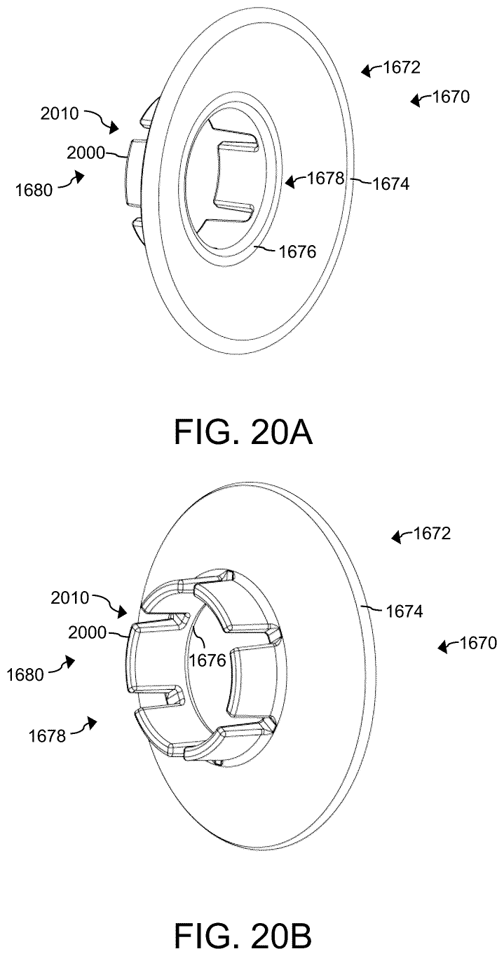

FIGS. 20A and 20B are isolated isometric views of the fluid-collection ring 1670, shown in FIGS. 19A and 19B, from different perspectives to further illustrate the distal sides and proximal sides 1672, 1680, respectively. A plurality of tabs 2000 are attached to the inner edge 1676 of the fluid-collection ring 1670. The tabs 2000 are configured to mechanically engage the shaft 200 to mechanically secure the fluid-collection ring 1670 to the shaft 200. In some embodiments, the handle 1660 can include complementary tabs or ridges that are configured to mechanically engage the gaps 2010 between neighboring tabs 2000 for example to prevent rotation of the fluid-collection ring 1670 relative to the handle 1660 while allowing rotation of the fluid-collection ring 1670 relative to the shaft 200.

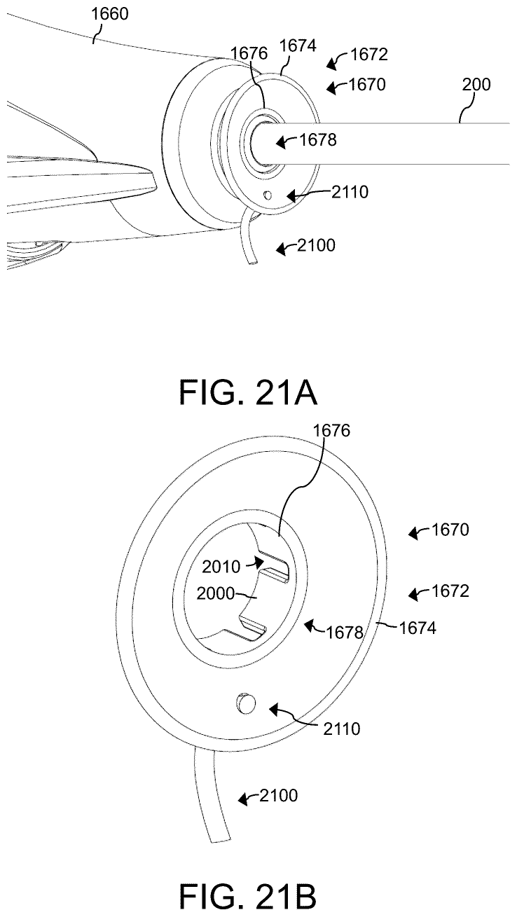

FIGS. 21A and 21B illustrate a fluid-collection ring 1670 having a drainage tube 2100 that is coupled to a fluid-drainage hole 2110 in the fluid-collection ring 1670. Other than the drainage tube 2100 and the fluid-drainage hole 2110, the fluid-collecting ring 1670 illustrated in FIGS. 21A and 21B can be the same as the fluid-collection ring 1670 shown in FIGS. 16, 17A, 17B, 17C, 18A, 18B, 19A, 19B, 20A, and/or 20B. The fluid-collection ring 1670 can be configured such that when the shaft 200 is oriented horizontally (or approximately horizontally such as within about 15 degrees of a horizontal plane), the fluid-drainage hole 2110 is located below the shaft 200 so as to be in position to collect fluids that may flow downward on the distal side 1672 of the fluid-collecting ring 1670 during a medical procedure.

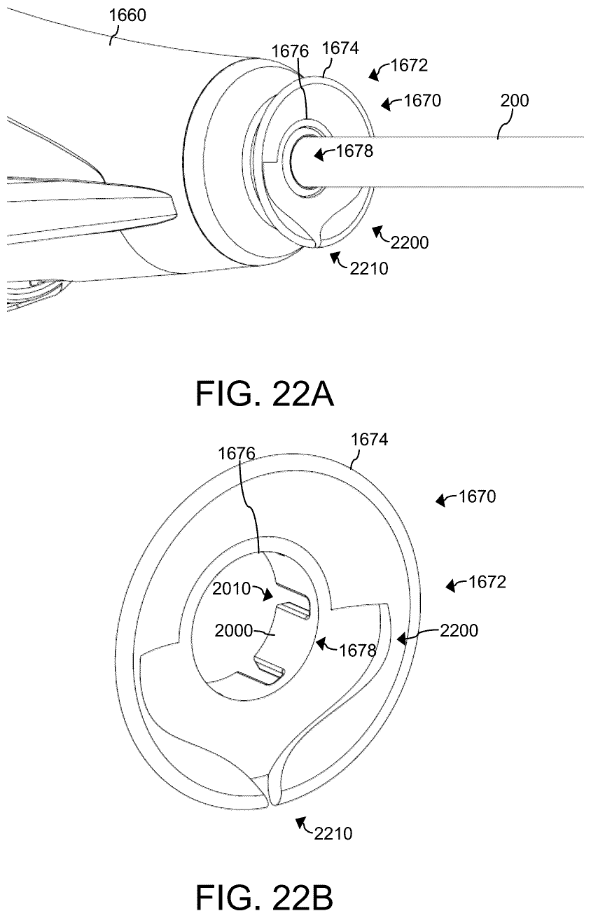

FIGS. 22A and 22B illustrate a fluid-collection ring 1670 having a fluid-drainage channel or recess 2200 defined in the distal side or surface 1672 of the fluid-collection ring 1670. The fluid-drainage channel or recess 2200 can extend to a fluid-drainage hole 2210 that can be defined in the outer edge 1674 of the fluid-collection ring 1670. The fluid-collection ring 1670 can be configured such that when the shaft 200 is oriented horizontally (or approximately horizontally such as within about 15 degrees of a horizontal plane), the fluid-drainage channel or recess 2200 and the fluid-drainage hole 2210 are located below the shaft 200 so as to be in position to collect fluids that may flow downward on the distal side 1672 of the fluid-collecting ring 1670 during a medical procedure.

Other than the fluid-drainage channel or recess 2200 and the fluid-drainage hole 2210, the fluid-collecting ring 1670 illustrated in FIGS. 22A and 22B can be the same as or combined with the fluid-collection ring 270 shown in FIGS. 16, 17A, 17B, 17C, 18A, 18B, 19A, 19B, 20A, 20B, 21A, and/or 21B.

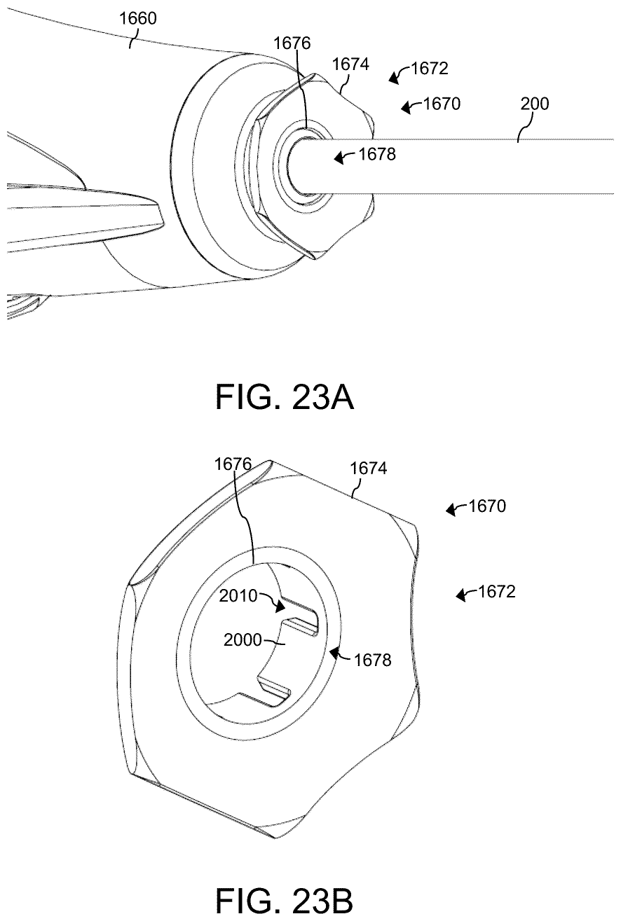

FIGS. 23A and 23B illustrate a fluid-collection ring 1670 having an outer edge 1674 that is hexagonal. The outer edge 1674 can be another shape, such as another polygonal shape, in other embodiments.

Other than the shape of the outer edge 1674, the fluid-collecting ring 1670 illustrated in FIGS. 23A and 23B can be the same as or combined with the fluid-collection ring 270 shown in FIGS. 16, 17A, 17B, 17C, 18A, 18B, 19A, 19B, 20A, 20B, 21A, 21B, 22A, and/or 22B.

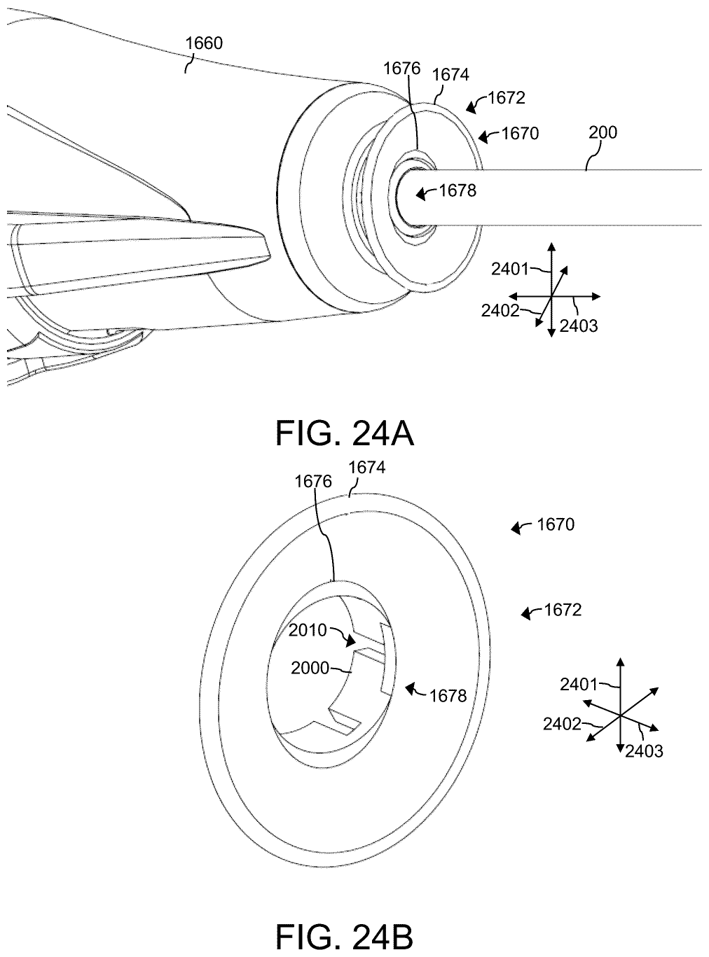

FIGS. 24A and 24B illustrate a fluid-collection ring 1670 having an oval shape. The fluid-collection ring 1670 is elongated with respect to a first axis 2401 compared to with respect to a second axis 2402 that is orthogonal to the second axis 2402. The shaft 200 extends along or parallel to a third axis 2403 that is orthogonal to the first and second axes 2401, 2402. Thus, axes 2401-2403 are mutually orthogonal.

Other than the shape of the fluid-collection ring 1670, the fluid-collecting ring 1670 illustrated in FIGS. 24A and 24B can be the same as or combined with the fluid-collection ring 270 shown in FIGS. 16, 17A, 17B, 17C, 18A, 18B, 19A, 19B, 20A, 20B, 21A, 21B, 22A, 22B, 23A, and/or 23B.

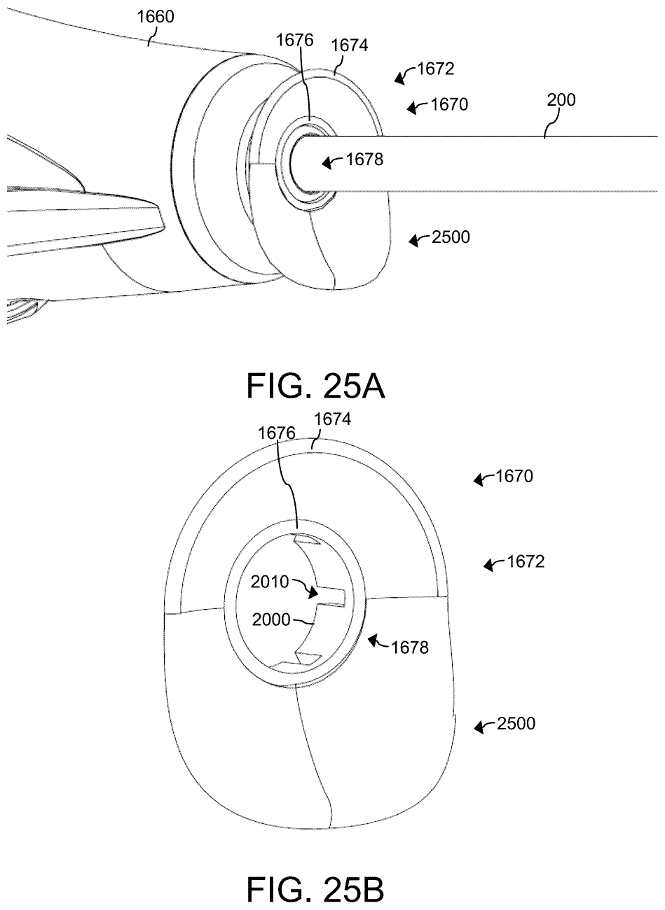

FIGS. 25A and 25B illustrate a fluid-collection ring 1670 having an oval shape and a flared portion 2500. The flared portion 2500 can function as a channel that directs fluids downward and away from the handle 1660. The fluid-collection ring 1670 can be configured such that when the shaft 200 is oriented horizontally (or approximately horizontally such as within about 15 degrees of a horizontal plane), the flared portion 2500 is located below the shaft 200 so as to be in position to direct fluids downward and away from the handle 1660 during a medical procedure.

Other than the shape of the fluid-collection ring 1670 and the flared portion 2500, the fluid-collecting ring 1670 illustrated in FIGS. 25A and 25B can be the same as or combined with the fluid-collection ring 1670 shown in FIGS. 16, 17A, 17B, 17C, 18A, 18B, 19A, 19B, 20A, 20B, 21A, 21B, 22A, 22B, 23A, 23B, 24A, and/or 24B.

The invention should not be considered limited to the particular embodiments described above, but rather should be understood to cover all aspects of the invention as fairly set out in the attached claims. Various modifications, equivalent processes, as well as numerous structures to which the invention may be applicable, will be apparent to those skilled in the art to which the invention is directed upon review of this disclosure. The claims are intended to cover such modifications and equivalents.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. An ultrasound probe comprising:
a shaft having a proximal end, a tip, and a length measured between the proximal end and the tip with respect to an axis;
a plurality of channels defined in the shaft and extending from the proximal end of the shaft along at least a portion of the length of the shaft, the plurality of channels including an ultrasound channel and a balloon channel;
one or more ultrasound transducers disposed in the ultrasound channel; and
an offset balloon attached to a distal portion of the shaft and disposed at a predetermined distance from the ultrasound transducer(s), the offset balloon fluidly coupled to the balloon channel to receive or withdraw a fluid to adjust an inflation state of the offset balloon, the offset balloon having a deflated state and an inflated state,
wherein:
in the inflated state the offset balloon has an inflated size that is larger than a target anatomical opening such that an inflated offset balloon is configured to mechanically engage the target anatomical opening when the shaft is retracted with respect to the target anatomical opening, and
the ultrasound transducer(s) is/are aligned with respect to a target volume when the inflated offset balloon mechanically engages the target anatomical opening.

2. The ultrasound probe of claim 1, further comprising a coating on the offset balloon.

3. The ultrasound probe of claim 2, wherein the coating comprises a medication, an antibacterial agent, and/or a lubricant.

4. The ultrasound probe of claim 1, wherein the offset balloon is attached to a distal end of the shaft.

5. The ultrasound probe of claim 1, wherein:
the offset balloon is a first offset balloon,
the balloon channel is a first balloon channel,
the predetermined distance is a first predetermined distance,
the plurality of channels includes a second balloon channel,
the fluid is a first fluid, and
the ultrasound probe further comprises a second offset balloon attached to the distal portion of the shaft and disposed at a second predetermined distance from the ultrasound transducer(s), the second offset balloon fluidly coupled to the second balloon channel to receive or withdraw a second fluid to adjust the inflation state of the second offset balloon, the second offset balloon having the deflated state and the inflated state, the second balloon in the inflated state having a size that is larger than the target anatomical opening,
whereby an alignment with respect to the target volume can be adjusted by transitioning the first offset balloon to the inflated state or by transitioning the second offset balloon to the inflated state.

6. The ultrasound probe of claim 1, further comprising:
a handle attached to the proximal end of the shaft; and
a fluid-collection ring disposed on a distal side of the handle, the fluid-collection ring having an inner edge and an outer edge, the inner edge defining a hole through which the shaft extends, a distal side of the fluid-collection ring having a concave shape and configured to direct a liquid away from the handle.

7. The ultrasound probe of claim 6, wherein the inner edge and the shaft define a fluid-collection channel and the medical device further comprises a fluid-collection reservoir that is fluidly coupled to the fluid-collection channel.

8. The ultrasound probe of claim 6, wherein a drainage hole is defined through a proximal side and the distal side of the fluid-collection ring and a drainage tube is fluidly coupled to the drainage hole on the proximal side of the fluid-collection ring.

9. The ultrasound probe of claim 6, wherein:
the axis is a first axis,
the fluid-collection ring is elongated with respect to a second axis compared to with respect to a third axis, and
the first, second, and third axes are mutually orthogonal.

10. The ultrasound probe of claim 1, further comprising:
a handle attached to the proximal end of the shaft; and
a fluid-collection ring disposed on a distal side of the handle, the fluid-collection ring having an inner edge, an outer edge, and a fluid-collection channel defined on a distal side of the fluid-collection ring.

11. The medical device of claim 10, wherein the fluid-collection channel is defined only on a distal surface of the fluid-collection ring.

12. The medical device of claim 11, wherein the fluid-collection channel extends to a hole defined in the outer edge of the fluid-collection ring.

13. The medical device of claim 12, wherein the hole is oriented at a bottom of the fluid- collection channel when the shaft is oriented parallel to a horizontal axis.

14. An ultrasound probe comprising:
a shaft having a proximal end, a tip, and a length measured between the proximal end and the tip with respect to an axis;
a plurality of channels defined in the shaft and extending from the proximal end of the shaft along at least a portion of the length of the shaft, the plurality of channels including an ultrasound channel and a balloon channel;
one or more ultrasound transducers disposed in the ultrasound channel; and
an offset balloon attached to a middle portion of the shaft and disposed at a predetermined distance from the ultrasound transducer(s), the offset balloon fluidly coupled to the balloon channel to receive or withdraw a fluid to adjust an inflation state of the offset balloon, the offset balloon having a deflated state and an inflated state,
wherein:
in the inflated state the offset balloon has an inflated size that is larger than a target anatomical opening such that an inflated offset balloon is configured to mechanically engage the target anatomical opening when the shaft is inserted into the target anatomical opening, and
the ultrasound transducer(s) is/are aligned with respect to a target volume when the inflated offset balloon mechanically engages the target anatomical opening.

15. The ultrasound probe of claim 14, further comprising a coating on the offset balloon.

16. The ultrasound probe of claim 15, wherein the coating comprises a medication, an antibacterial agent, and/or a lubricant.

17. The ultrasound probe of claim 14, wherein:
the offset balloon is a first offset balloon,
the balloon channel is a first balloon channel,
the predetermined distance is a first predetermined distance,
the plurality of channels includes a second balloon channel,
the fluid is a first fluid, and
the ultrasound probe further comprises a second offset balloon attached to a distal portion of the shaft and disposed at a second predetermined distance from the ultrasound transducer(s), the second offset balloon fluidly coupled to the second balloon channel to receive or withdraw a second fluid to adjust the inflation state of the second offset balloon, the second offset balloon having the deflated state and the inflated state.

18. The ultrasound probe of claim 14, further comprising:
a handle attached to the proximal end of the shaft; and
a fluid-collection ring disposed on a distal side of the handle, the fluid-collection ring having an inner edge and an outer edge, the inner edge defining a hole through which the shaft extends, a distal side of the fluid-collection ring having a concave shape and configured to direct a liquid away from the handle.

19. A method for performing thermal therapy, comprising:
inserting a distal portion of a shaft of an ultrasound probe into a target anatomical opening, the shaft having a proximal end, a tip, and a length measured between the proximal end and the tip with respect to an axis, the ultrasound probe further comprising:
a plurality of channels defined in the shaft and extending from the proximal end of the shaft along at least a portion of the length of the shaft, the plurality of channels including an ultrasound channel and a balloon channel;
one or more ultrasound transducers disposed in the ultrasound channel; and
an offset balloon attached to the distal portion of the shaft, the offset balloon fluidly coupled to the balloon channel to receive or withdraw a fluid to adjust an inflation state of the offset balloon, the offset balloon in a deflated state;
inflating the offset balloon while the distal portion of the shaft, including the offset balloon, is disposed in the target anatomical structure;
retracting the shaft until the offset balloon mechanically engages the target anatomical opening, the offset balloon in an inflated state and having an inflated size that is larger than the target anatomical opening, the ultrasound transducer(s) disposed at a predetermined distance from the offset balloon;
aligning the ultrasound transducer(s) with respect to a target volume; and
applying ultrasound energy, with the ultrasound transducers, to the target volume while the ultrasound transducers are aligned with the target volume.

20. The method of claim 19, wherein:
the offset balloon is a first offset balloon,
the balloon channel is a first balloon channel,
the predetermined distance is a first predetermined distance,
the plurality of channels includes a second balloon channel,
the fluid is a first fluid, and
the method further comprises:
after retracting the shaft until the first offset balloon mechanically engages the target anatomical opening, determining that the ultrasound transducer(s) is/are not aligned with respect to the target volume;
deflating the first offset balloon;
inflating a second offset balloon attached to the distal portion of the shaft, the second offset balloon fluidly coupled to the second balloon channel to receive or withdraw a second fluid to adjust the inflation state of the second offset balloon; and
retracting the shaft until the second offset balloon mechanically engages the target anatomical opening, the second offset balloon in the inflated state and having an inflated size that is larger than the target anatomical opening, the ultrasound transducer(s) disposed at a second predetermined distance from the second offset balloon,
wherein the ultrasound transducer(s) is/are aligned with respect to the target volume at the second predetermined distance from the second offset balloon.
